(19) United States
(12) Patent Application Publication
(10) Pub. No.: US 2015/0196638 A1
(43) Pub. Date: Jul. 16, 2015

(54) SYSTEM AND METHOD FOR USING ULTRASOUND-STIMULATED MICROBUBBLE EXPOSURES TO INDUCE CERAMIDE ACCUMULATION IN ENDOTHELIAL AND TUMOR CELLS

(71) Applicant: SUNNYBROOK HEALTH SCIENCES CENTRE, Toronto (CA)

(72) Inventors: Gregory J. Czarnota, Oakville (CA); Azza Almahrouki, Scarborough (CA); Raffi Karshafian, Richmond Hill (CA); William Tyler Tran, Toronto (CA); Peter Nicholas Burns, Toronto (CA); Christina Kim, Toronto (CA); Anoja Giles, North York (CA); Emily Wong, Toronto (CA)

(21) Appl. No.: 14/413,623

(22) PCT Filed: Jul. 8, 2013
(86) PCT No.: PCT/CA2013/050526

§ 371 Date: Jan. 8, 2015

Related U.S. Application Data
(60) Provisional application No. 61/669,102, filed on Jul. 8, 2012.

(51) Int. Cl. A61K 41/00; A61N 5/10; A61N 7/00

(52) U.S. Cl. CPC A61K 41/0028; A61N 7/00; A61N 5/10; A61N 2007/0039

(57) ABSTRACT

A system and method for using ultrasound and a microbubble agent to induce ceramide accumulation in a target region including a population of cells associated with a tumor. An ultrasound system is directed to expose a target region in a patient, to which a microbubble agent has been provided, to an ultrasound exposure sufficient to alter gene expressions in cells in the target region so as to induce an accumulation of ceramide in the cells.

### CROSS-REFERENCE TO RELATED APPLICATIONS

This application claims the benefit of U.S. Provisional Patent Application Ser. No. 61/669,102 filed on Jul. 8, 2012, and entitled “SYSTEM AND METHOD FOR USING ULTRASOUND-STIMULATED MICROBUBBLE EXPOSURES TO INDUCE CERAMIDE ACCUMULATION IN ENDOTHELIAL AND TUMOR CELLS.”

### STATEMENT REGARDING FEDERALLY SPONSORED RESEARCH

This invention was made with government support under W81XWH-08-1-0400 awarded by the U.S. Army Medical Research & Material Command. The government has certain rights in the invention.

### BACKGROUND OF THE INVENTION

The field of the invention is systems and methods for ultrasound. More particularly, the invention relates to systems and methods for mediating ceramide accumulation in endothelial cells using ultrasound.

Tumors rely on blood vessels for survival. Tumor responses to radiotherapy can be affected by pro-angiogenic factors that protect endothelial cells, contributing to tumor radioresistance. Radiation can also provoke an up-regulation of vascular endothelial growth factor (“VEGF”) to protect endothelial cells against apoptosis, which has been demonstrated to occur within twenty-fourhours after radiation. These findings suggest that targeting vascular endothelial cells can be an effective strategy to enhance tumor response to radiation.

In addition to angiogenesis, the survival of cancer cells is often further complicated by the presence of nearby healthy tissue. This often necessitates the use of low doses of radiation in order to avoid radiation toxicity effects. Hence, treatments that can maximize the effects of radiation and yet spare healthy tissue are necessary. The search for these new treatments has led to the development of numerous radiation-enhancing cancer-fighting strategies, which involve the combination of radiotherapy with other therapeutic modalities. These include the inhibition of epidermal growth factor receptors and new anti-angiogenic drugs.

Anti-vascular agents that target endothelial cells are primarily focused on inhibition of angio-regulators such as VEGF, angiogenin, and thrombin. Antivascular pharmacological agents have been successful in preclinical trials, although there are several limitations to their application as a monotherapeutic approach. These limitations include the requirement to administer a high dose within a prolonged treatment course in order to achieve optimal therapeutic efficacy and obstacles in targeting only tumor vasculature while sparing normal, healthy vasculature. Moreover, antivascular pharmacological agents can result in undesirable side effects, including anorexia, constipation, dyspnea, fatigue, headache, pain and hypokalemia in addition to rebound tumor growth after the cessation of therapy

Thus, there remains a need for more effective anti-angiogenic mechanisms in cancer treatment, such as anti-angiogenic mechanisms that do not require pharmaceutical treatments with potential undesirable side effects or mechanisms that promote vascular regrowth.

### SUMMARY OF THE INVENTION

The present invention overcomes the aforementioned drawbacks by providing a method for using ultrasound and a microbubble agent to induce a therapeutic accumulation of ceramide in a targeted cell population, such as a tumor. The method includes directing an ultrasound system to expose a target region in a patient to which a microbubble agent has been provided to an ultrasound exposure sufficient to alter gene expressions in cells in the target region so as to induce an accumulation of ceramide in the cells.

It is an aspect of the invention to provide a method for controlling an ultrasound system to induce a therapeutic accumulation of ceramide in a target region of a subject by directing the ultrasound system to expose a target region in the subject to which a microbubble agent has been provided to an effective exposure of ultrasound sufficient to induce a therapeutic accumulation of ceramide in the target region.

It is another aspect of the invention to provide a method for inducing a therapeutic accumulation of ceramide in a target region of a subject by administering an effective amount of a microbubble agent to a subject and directing an ultrasound system to expose a target region in the subject in which the microbubble agent is present to an effective exposure of ultrasound.

It is yet another aspect of the invention to provide a method for treatment of cancer in a subject by administering an effective amount of a microbubble agent to the subject and providing an effective exposure of ultrasound to the subject sufficient to interact with the microbubble agent and cause vascular disruption in endothelial cells associated with a cancerous tumor. The method can further include providing an effective dose of an energy source, such as radiation, thermal, or electromagnetic, to the cancerous tumor sufficient to increase the vascular disruption in the endothelial cells associated with the cancerous tumor.

It is yet another aspect of the invention to provide an intravenously administrable composition for increasing ceramide in a population of cells associated with a tumor, comprising a microbubble agent having a concentration in the range of about 1.8×10̂4 microbubbles per milliliter to about 5.4×10̂8 microbubbles per milliliter, which when exposed to an effective exposure of ultrasound induces a therapeutic accumulation of ceramide in a subject.

It is yet another aspect of the invention to provide an ultrasound system for generating an anti-angiogenic and anti-tumor bioeffect in a population of cells located within a target region. Such a system includes an ultrasound transducer configured to deliver ultrasound waves to a target region and a control system in communication with the ultrasound transducer. The control system is configured to set an effective ultrasound exposure sufficient to induce disruption of an effective amount of a microbubble agent such that the disruption of the microbubble agent is capable of generating an anti-angiogenic or anti-tumor bioeffect in cells; and to direct the ultrasound transducer to produce ultrasound waves at the set ultrasound exposure such that the ultrasound waves are delivered to the target region.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a block diagram of an example of an ultrasound system configured to alter a gene expression in cells in a target region, such as to alter gene expressions so as to induce ceramide accumulation in the cells in the target region;

FIG. 2 shows results of cell death assessments. (A) Representative haematoxylin and eosin (H&E) staining and (B) corresponding ISEL stained sections of PC3 cell prostate tumors treated with radiation and/or ultrasound-activated microbubbles. Columns represent 0, 2, and 8 Gy of radiation exposure from left to right. Rows indicate no (Nil), low concentration microbubble exposure (LMB) and high concentration microbubble exposure (H MB) from top to bottom, respectively. (C) Data from low power microscope images of whole tumour sections quantifying the extent of ISEL staining for each treatment demonstrating enhanced tumour cell death when radiation (4±2% ISEL+staining, 2Gy) is combined with ultrasound-activated microbubble treatment (10±4% ISEL+staining, LMB) resulting in 40±10% cell death when combined. (D) Data on apoptotic cells detected from stained sections based on morphological observation at high power. (scale bar=2 mm).

FIG. 3 shows power Doppler ultrasound imaging and high magnification immunohistochemical data for radiation and ultrasound treatments. (A) Power Doppler (PD) images, ISEL high magnification data (ISEL) and von Willebrand Factor staining for vasculature (VWF). Columns indicate data for no treatment (Nil), ultrasound-activated microbubbles (MB), 8 Gy radiation (XRT) and microbubble and radiation treatments combined (MB XRT). (B) Triple staining for endothelial cell apoptosis with TUNEL+apoptotic nuclei, CD31 vascular delineation and DAPI nuclear staining in tumour samples treated with HMB (scale bar=20 microns).

FIG. 4 shows analyses of cell death for treatments with targeted microbubbles and treatments with microbubbles in the presence of bFGF.

FIG. 5 shows quantitative analysis of cell death in response to microbubble exposure with different radiation doses. Percentage ISEL+ staining from 4 tumors per group is shown with different microbubble concentrations administered to mice. For microbubble concentrations Nil indicated no treatment, 0.01L and 0.1L indicate dilutions of LMB (L) and (H) indicates the HMB used. Different radiation doses include 0, 2 and 8 Gy as labeled.

FIG. 6 shows quantitative analysis of cell death in response to timing between microbubble exposure and radiation treatment. (A) Decrease in micro-power Doppler data measured vascular index with microbubbles and combined treatment (Nil—no treatment imaged before and 24 hours later; MB—treatment with microbubbles only (low concentration) and sacrifice of mice at the indicated times after microbubble exposure (0, 3, 6, 12 and 24 hours); MBXRT—treatment with microbubbles and interval time as indicated between subsequent radiation treatment (8 Gy). (B) Resulting ISEL+cell death corresponding to treatments as described in (A).

FIG. 7 shows tumor growth delay data for single treatments. Legend: Nil—no treatment (solid line, black circles), MB—ultrasound-activated microbubble treatment (dashed line, white triangles), 2 Gy-2 Gy x-ray radiation (dashed line, black triangles), 8Gy-8Gy x-ray radiation (solid line, black squares), MB2Gy—combined treatment (solid line, white squares), MB 8 Gy—combined treatment (solid line, white circles).

FIG. 8 shows response assessments for multiple fraction experiments. (A) Survival data are indicated in Kaplan-Meyer survival curves for cohorts of mice treated with 2 Gy fractions (24 Gy in 12 fractions over three weeks (BED(10)=28.8), 2 Gy fractions with two ultrasound-stimulated-microbubble treatments weekly, 3 Gy fractions (45 Gy in 15 fractions over three weeks (BED(10)=58.5), and ultrasound-stimulated-microbubble treatments weekly (twice weekly for three weeks). (B) Ki-67 analysis by counting of representative tumor sections.

FIG. 9 shows in vivo ceramide staining data. (A) Quantification of ceramide immunohistochemistry staining of sections of PC3 prostate tumors treated with radiation and/or ultrasound-activated microbubbles. Labels indicate non (Nil), low (Low) and high concentration microbubble exposure (High) and radiation doses (0, 2 or 8 Gy). (B) Quantification of ceramide immunohistochemistry staining for experiments with S1P.

FIG. 10 shows quantification of apoptosis with ceramide cell death inhibition for PC3 prostate tumors treated with radiation and/or ultrasound-activated microbubbles in the presence of S1P. Labels indicate no (Nil), and high-microbubble exposure (High) and radiation doses (0, 2 or 8 Gy) in the presence of sphingosine-1-phosphate (S1P).

FIG. 11 shows survival assays of wild-type, mutant and S1P-treated astrocytes treated with combination of ultrasound and microbubbles (A) and ultrasound alone (B) as well as for HUVECs treated with S1P then exposed to radiation alone or ultrasound/microbubbles alone (C) versus radiation/ultrasound/microbubbles (D).

FIG. 12 shows graphs of 24 hour response monitoring of tumor vasculature index using Doppler ultrasound for HT-1376 bladder cancer tumors: (A) combination therapy with LMB and (B) combination therapy with HMB.

FIG. 13 shows long-term response monitoring of tumor vasculature in HT-1376 bladder cancer xenograft mouse model. (A) Tumors treated with varying doses of radiation alone, (B) radiation treatment combined with LMB, (C) radiation treatment combined with HMB.

FIG. 14 shows effects of ultrasound-activated microbubbles and radiation on the growth of HT-1376 bladder cancer xenografts in SCID mice. Mice were divided into treatment groups: (A) no treatment or radiation alone, (B) LMB alone or combined with radiation, and (C) HMB alone or combined with radiation.

FIG. 15 shows (A) H&E staining of whole PC3 xenograft tumor sections treated with 0, 2 & 8 Gy or with a combination of radiation and ultrasound-stimulated microbubbles (−MB indicates no exposure to ultrasound-stimulated microbubbles, +MB indicates treatment with ultrasound-stimulated microbubbles. (B) Sections adjacent to those in (A) were labeled with ISEL to illustrate areas of cell death. Scale bars=1 mm. (C) Quantified analyses of ISEL images indicating an increased level of cell death with the combined treatments. Mann-Whitney test used to calculate the P values and * symbols indicate where P-values are less than 0.05. (D) Clonogenic assay results illustrated a significant decrease in cellular survival of treated tumor cells when compared to the untreated samples.

FIG. 16 shows detection of cellular proliferation using Ki67 as a marker. (A) More labeled nuclei were observed in the controls than in the treated samples (radiation +/− microbubbles). This indicated a decreased proliferation specifically with the combined treatment of ultrasound-stimulated microbubbles and radiation, where a significant difference was found P<0.024 (see (B−*)). (B) The number of positively stained nuclei counted in whole sections and the number of the labeled cells/mm² were calculated and plotted. A Mann-Whitney test was used to calculate the P values. The magnification bar represents 25 nm.

FIG. 17 shows vessel integrity detected using immunohistochemical labeling of factor VIII in PC3 xenograft sections. (A) Micrographs of sections from tumors not treated with microbubbles (−MB/upper panel) and tumors treated with microbubbles (+MB/lower panel). (B) Blood vessel leakage became more evident and significant when the ultrasound activated microbubbles were combined with radiation dose of 8Gy. Statistical analyses indicated a P<0.029. The magnification bar represents 50 nm.

FIG. 18 shows angiogenesis assessment in PC3 xenograft sections using immunohistochemical CD31 labeling. (A) Micrographs of tumor sections illustrating labeling of endothelial cells treated with different conditions, where fewer labeled vessels were observed. (B) Decreased vascularization was observed following treatments of 8Gy (P<0.043), MB+2Gy (P<0.032) and MB+8Gy (P<0.01). Scale bar=25 μm.

FIG. 19 shows assessment of angiogenesis signaling in PC3 xenograft sections following VEGF immunohistochemical analyses. (A) Micrographs of tumor sections illustrating labeling of endothelial cells treated with different conditions, where fewer labeled vessels were observed. (B) A significant signaling increase was observed following combined (MB+8Gy) treatments (P<0.032). Scale bar=50 μm.

FIG. 20 shows hypoxia staining in PC3 xenograft sections using PHD2 immunohistochemical analysis of tumor sections. (A) Labeled sections illustrated an increased staining with the combined treatments. (B) Statistical analyses revealed a significant change when comparing the controls to 8Gy (P<0.05), to MB+2Gy (P<0.008), or to MB+8Gy (P<0.012). Scale bar=25 μm.

FIG. 21 shows DNA damage in PC3 xenograft sections. Immunofluorescence analyses of gamma H2AX (upper two panels) and overlay with Dapi as a counter stain (lower two panels). (B) Increased labeling was observed with all the treatments with P<0.029 (MB and MB+2Gy) and P<0.014 (2Gy, 8Gy and MB+8Gy). Scale bar=30 μm.

FIG. 22 shows ceramide labeling of PC3 xenograft sections. (A) Ceramide labeling increased in intensity and distribution with the combined treatments compared to single treatments. (B) Labeling analyses using ImageJ indicated a significant difference when comparing the labeling of the different treatment groups to the control with P=0011; with either 2Gy alone or combined, P=0005; with either US/MB or 8Gy alone, and P=0002 with US/MB+8Gy. A Mann-Whitney test was used to calculate the P values (scale bar=50 μm).

### DETAILED DESCRIPTION OF THE INVENTION

The term “administration” and variants thereof (e.g., “administering” a composition) in reference to an inventive composition means providing the composition to an individual in need of treatment.

As used herein, the term “composition” is intended to encompass a chemical composition comprising the specified ingredients, as well as any product which results, directly or indirectly, from combining the specified ingredients.

The term “subject” as used herein refers to an animal, preferably a mammal, most preferably a human, who has been the object of treatment, observation, or experiment. Furthermore, the terms “human,” “patient,” and “subject” are used interchangeably herein.

The term “effective amount” as used herein means that amount of composition that is sufficient to elicit the biological or medicinal response in a tissue, system, animal, or human that is being sought by a researcher, veterinarian, medical doctor, or other clinician.

The term “effective exposure” as used herein means that exposure of ultrasound that, when combined with the effective amount of composition, elicits the biological or medicinal response in a tissue, system, animal, or human that is being sought by a researcher, veterinarian, medical doctor, or other clinician. In one embodiment, exposing the effective amount of composition to the effective exposure of ultrasound induces a therapeutic accumulation of ceramide in the subject. In another embodiment, exposing the effective amount of composition to the effective exposure of ultrasound elicits vascular disruption in a targeted cell population in the subject.

The term “therapeutic accumulation” of ceramide as used herein means that accumulated amount of ceramide that results in apoptosis or other cellular death in a tumor or other targeted cell population.

The term “effective dose” as used herein means that dose of radiation that, when combined with the effective amount of composition and effective exposure of ultrasound, elicits the biological or medicinal response in a tissue, system, animal, or human that is being sought by a researcher, veterinarian, medical doctor, or other clinician.

“Treating” or “treatment” of any condition or disorder refers, in one embodiment, to ameliorating the condition or disorder (i.e., arresting or reducing the development of the condition or at least one of the clinical symptoms thereof). In another embodiment “treating” or “treatment” refers to ameliorating at least one physical parameter, which may not be discernible by the subject. In yet another embodiment, “treating” or “treatment” refers to modulating the condition or disorder, either physically, (e.g., stabilization of a discernible symptom), physiologically, (e.g., stabilization of a physical parameter), or both.

The present invention is based on the inventors' success in identifying synergistic combinations of certain compositions and effective exposures of ultrasound useful in treating cancer in the living body. Accordingly, a first aspect of the invention is directed to synergistic combinations of compositions such as microbubble agents and effective exposures of ultrasound that induce therapeutic accumulations of ceramide in a target region of a subject in connection with cancer treatment. Such combinations can further be combined with effective doses of an energy source, such as a radiation source, thermal source, or electromagnetic source sufficient to increase the therapeutic accumulation of ceramide to an increased therapeutic accumulation of ceramide.

Ultrasound-stimulated microbubbles have been used to induce temperature increases in tissue, to increase permeability of cellular membranes to influxes of ions through membrane ion channels, and to induce the production of free radicals that can be damaging and induce cell death. Microbubbles have also been used as drug and gene delivery agents for cancer therapies, where targeted microbubbles are used as delivery vehicles. However, unlike these previous uses of microbubble agents, the system and method of the present invention utilize microbubble agents to modify gene expression levels in different cell types. Notably, the system and method of the present invention enable the use of ultrasound-stimulated microbubbles, whether non-targeted or targeted, to induce the accumulation of ceramide in different cell types, such as endothelial cells.

This accumulation of ceramide can be utilized as a cancer treatment in isolation, or can be combined with other cancer treatment regimes. For instance, the accumulation of ceramide can beneficially increase the radiosensitivity of certain cell types, thereby making radiation treatment protocols more effective against otherwise radioresistant tumors. When using targeted microbubbles, such as vascular endothelial growth factor receptor (“VEGFR2”)-targeted microbubbles, the ceramide accumulation, and resultant increased radiosensitivity, can be localized to cancerous tissues, thereby increasing the efficacy of the radiation treatment while reducing the effect of excess radiation to normal tissues surrounding the treated tumor.

Thus, a system and method for altering the gene expression of a cell using ultrasound in conjunction with microbubbles is provided. The system and method are capable of up regulating genes involved in cellular apoptosis, notably those genes that play a role in ceramide-induced apoptotic signaling pathways. Notably, the inventors have discovered that ceramide accumulation in cells can be induced using ultrasound-stimulated microbubbles when operating the ultrasound system to produce an effective exposure and when an effective concentration of microbubbles are present in the target region. By altering the gene expression of cells using ultrasound-stimulated microbubbles such that ceramide accumulation occurs, the radioresistance and chemoresistance of the treated cells can be significantly reduced, thereby enhancing the treatment of commonly radiosensitive or chemosensitive cells and making commonly radioresistive cells radiosensitive or chemoresistive cells chemosensitive.

As noted above, the therapeutic effect of ceramide accumulation induced in targeted cells by ultrasound-stimulated microbubble may be complemented, or augmented, by other forms of energy capable of inducing ceramide accumulation. Examples of other forms of energy include radiation, as may be used in radiotherapy; thermal energy, as may be used in thermal ablation therapies; and electromagnetic energy, as may be used in radio frequency ablation therapies. The therapeutic effect of the present invention is additive; therefore, when other forms of energy are used to complement, or augment, the ceramide accumulation induced by the present invention, these other forms of energy may be used before, after, or contemporaneously with the ultrasound-stimulated microbubbles.

Ultrasound-stimulated microbubble exposures are used to induce changes in gene expression. Genes that are up regulated by this process include sphingomyelin phosphodiesterase 2 (“SMPD2”), which is a membrane-bound enzyme related to membrane damage signaling; cytochrome C oxidase (“COX6B1”) and mitogen activated protein kinase kinase 1 (“MAP2K1”), which are genes related to downstream cell death signaling; and UDP glycosyltransferase 8 (“UGT8”), which is involved in lipid biogenesis and repair. Additionally, Caspase 9 genes, which are also involved in lipid biogenesis and repair, were elevated when ultrasound-stimulated microbubbles were combined with radiation treatment. Overall, these genes code for proteins that are involved in ceramide-regulated apoptosis pathways. Thus, ultrasound-stimulated microbubble exposures in accordance with the systems and methods of the present invention are effective at inducing ceramide accumulation in targeted cells. While increases in sphingomyelin phosphodiesterase 1 (“SMPD1”) have not been observed by the inventors to date, changes in intracellular locations of SMPD1 were observed following ultrasound-stimulated microbubble exposures.

Although activation of the ceramide signaling pathway, which results in an accumulation of ceramide in the targeted cells, is contemplated to be the dominant mechanism resulting in the radiosensitization of cells treated with ultrasound-stimulated microbubbles, other potential cellular mechanisms include mechanical perturbation leading directly to endothelial cell apoptosis, cytokine stimulation, and changes in ionic environment caused by vascular disruption.

For instance, microbubbles sequestered to the interior lumen of tumor vasculature can induce stress effects to the endothelium. When driven by ultrasound, microbubble disruption can result in damage to the endothelium through shear stress. This event can subsequently initiate gross vascular reorganization within the tumor compartment. Vessel phenotypes found within the tumor compartment are typically porous, leaky, and are architecturally entangled and premature. This poses a problem in the delivery of oxygen, a major radiation-enhancing molecule. In combination with cytotoxic therapies, vascular targeting agents are showing promise in tumor control by breaking the inter-reliance of cancer cells and endothelial cells in tumors. The present invention implements a biophysical targeting model to abrogate endothelial cells by the way of ultrasound-driven microbubbles. When used in combination with radiation, ultrasound-mediated microbubble treatment also enhances tumor killing.

Radiation treatments present several limitations in achieving optimal therapeutic outcomes because of tumor cell heterogeneity. As a result, many regimens require varying doses of radiation in order to control tumor growth and also to make a significant effect on the tumor vasculature. As one example, the present invention overcomes these limitations by providing tumor control with the delivery of only 2 Gy of radiation to the tumor in combination with ultrasound-mediated microbubble treatment. When 2 Gy radiation was combined with low and high concentrations of ultrasound-driven microbubbles, vessel disruption was observed in tumors, as described below.

Vascular disrupting agents serve as a feasible model in combination with cytotoxic agents such as radiation because targeting vasculature is independent of tumor type and can overcome the obstacles that cytotoxic drugs have in combating cell heterogeneity. Furthermore, tumor cells are more likely to resist cytotoxic therapies because of their genetic instability, and thus targeting the endothelial cells can be beneficial because of its relative accessibility to vascular targeting agents, its low potential to disrupt normal tissue, and its ability to influence death on tumor clonogens that are dependent on vessels.

Ultrasound-driven microbubbles used as vascular disrupting agents can have many advantages for molecular-based targeting. For instance, VEGF-antibody based agents could potentially have unwanted effects on other biological pathways as VEGF expression is also found in certain types of hematopoietic and stromal cells. Therefore, the added advantage to using ultrasound-driven microbubbles to perturb endothelial cells is its disposition to be locally targeted, thereby minimizing the influence on other biochemical pathways found in distant physiological processes.

A wide variety of cell types can be affected using the system and method of the present invention, including endothelial cells generally; acute myeloid leukemia (“AML”) cells; prostate cancer cells, such as those embodied by the PC3 cell line; murine fibrosarcoma cells, such as those embodied by the KHT-C cell line; breast cancer cells, such as those embodied by the MDA-MB-231 cell line; and astrocytes. For example, survival assays of asmase +/+ and asmase −/− astrocytes treated with ultrasound-stimulated microbubbles in combination with radiation demonstrate a significantly enhanced level of cell death as compared to survival in response to radiation alone. By using ultrasound-stimulated microbubbles, ceramide accumulation is induced in astrocytes, thereby reducing their radioresistance. Experiments conducted by the inventors have shown that astrocyte survival was 56±2 percent in response to a 2 Gy radiation dose alone, 17±7 percent in response to ultrasound-stimulated microbubbles alone, and 5±2 percent, or less, in response to a 2 Gy radiation dose with ultrasound-stimulated microbubble treatment. These results show, for example, that lower radiation doses, such as 2 Gy, can become effective when the radioresistance of cells is decreased due to the effect of ultrasound-stimulated microbubbles on ceramide accumulation in the cells.

Thus, in some instances, treatment with ultrasound and microbubbles alone may not result in increases in the expression of genes involved with apoptosis, such as those involved with ceramide-induced apoptosis. In these instances, the ultrasound-stimulated microbubble treatment can be combined with a radiation treatment to achieve the desired alteration in gene expression. Forms of radiation treatment include external beam radiotherapy, brachytherapy, and radioisotope therapy. Although ultrasound-stimulated microbubble exposures alone are capable of inducing ceramide accumulation, much higher relative gene expression levels, and therefore higher ceramide accumulations, can be obtained when the ultrasound-microbubble treatments are combined with radiation exposures, or other treatments that may induce ceramide accumulation. In other instances, ceramide-induced apoptosis may be augmented with other forms of therapy, such as chemotherapy.

In the method of the present invention, a microbubble agent is administered to the patient such that microbubbles are introduced into the target region, or the patient has already been administered a microbubble agent. Microbubble agents often include microscopic lipid or protein shells encapsulating gaseous content such as fluorocarbon gas including perfluoropropane and octafluoropropane. The compressible property of microbubble agents allows them to be excited by ultrasound waves at low pressures for imaging, higher pressures for drug delivery, and very high pressures for non-invasive ultrasound surgery. Microbubble-based treatments with ultrasound can make use of such intravascular microbubbles, or can alternatively convert liquid micro-droplets into gas bubbles that can form interstitially. In addition, at higher powers, ultrasound can cause bubble formation de novo from dissolved gases or from nanometer-scaled droplets of liquids that can be converted to gases.

By way of example, the microbubble agent may include an intravenously provided microbubble agent or an exogenous material, such as perfluorocarbon liquid droplets. For instance, the microbubble agent may be the perflutren lipid microsphere agent marketed under the name DEFINITY® (Lantheus Medical Imaging; N. Billerica, Mass.). A period of time is allowed to pass such that an effective concentration of the microbubble agent is present in the target region over a period of exposure. By way of example, an effective amount includes an amount sufficient to provide concentrations ranging from about 1.8×10̂4 bubbles/ml to 5.4×10̂8 bubbles/ml, but will depend on the type of microbubble agent and exposure time. It is contemplated that a higher concentration of microbubble agent results in a more significant accumulation of ceramide.

The patient may be administered a bolus or a continuous infusion of microbubble agent. The microbubble agent may be targeted or non-targeted. An example of a targeted microbubble agent includes avidin-conjugated MicroMarker Target-Ready Agent (VisualSonics; Toronto, Ontario) used with biotinylated VEGFR2 antibody (Abcam; Cambridge, Mass.). It is contemplated that targeted microbubble agents will demonstrate a greater effect compared to untargeted microbubble agents because targeted microbubble agents are more likely to be in close proximity to endothelial cells, which will increase oscillation-induced mechanical changes to cells.

Ultrasound exposures are preferably provided when a desired microbubble agent concentration is present in the target region. Example microbubble concentrations may be in the range of 100-300 times greater than those used for diagnostic imaging purposes; however, concentrations at diagnostic doses can also be used. These lower concentrations exhibited a lesser bioeffect; however, these lower concentration treatments may require using a greater overall duty cycle to achieve the insonification of the microbubble agent sufficient to achieve ceramide accumulation.

When there is a desired or maximal microbubble concentration within target region, the target region is exposed to ultrasound with a set ultrasound exposure that is sufficient to generate an anti-angiogenic bioeffect, such as by altering gene expressions such that ceramide accumulation in the targeted cells is induced. The microbubble concentration in the target region will define the ultrasound parameters necessary to achieve an effective ultrasound exposure. Moreover, it is contemplated that a microbubble agent concentration in an effective concentration range should be achieved before the target region is exposed to ultrasound. This is because the total exposure is dependent on the number of microbubbles that burst in the target region, which is, in turn, dependent on the microbubble concentration and exposure time. In addition, the type of microbubble agent that is used will impact the ultrasound pressure and frequency parameters.

An effective exposure of ultrasound may be achieved using ultrasound parameters that may range, but are not limited to, frequencies from 25 kHz to 5 MHz, with peak negative pressures of 50 kPa to 2.5 MPa, depending on the type of microbubble agent used and treatment conditions. Other ultrasound parameters may include exposure durations of 50 ns to 5 minutes, pulse repetition frequencies in the range of about 60 Hz to 60 MHz, and a mechanical index in the range of about 0.1 to about 4.0. Ultrasound operated using these parameters will produce an ultrasound exposure that is effective at stimulating microbubbles such that ceramide accumulation occurs in cells in the target region to which the ultrasound exposure is directed.

By way of example, an effective exposure of ultrasound may include the following ultrasound parameters: a half maximum peak of the acoustic signal of −6 dB beam width of 31 mm and −3 dB beam width of 18 mm; a ten percent duty cycle; 16-cycle tone bursts; at center frequency of 500 kHz; a pulse repetition frequency of 3 kHz; a peak negative pressure of 570 kPa; and a mechanical index of 0.80. It is noted, however, that based on the ranges of ultrasound parameters provided above, a mechanical index in the range of about 0.1-4.0 may be effective for inducing a therapeutic ceramide accumulation in cells in the target region exposed to ultrasound.

A pulse repetition period of 0.333 milliseconds over 50 milliseconds corresponds to 150 periods of 16 cycle tone bursts, or 4.8 milliseconds (rounded to 5 milliseconds). This 5 millisecond time can be selected to occur every two seconds to permit blood vessels to refill with microbubbles during a treatment time. A time interval, such as one on the order of 1950 milliseconds, between subsequent ultrasound exposures can be used to minimize biological heating in the target region during ultrasound exposures. The above ultrasound parameters may be controlled through information derived from real time ultrasound imaging. Because the ultrasound exposure is directed to the target region, areas outside of the target region remain substantially unaffected by the treatment.

The target region may optionally be exposed to an effective dose of radiation as well. Combined ultrasound-activated microbubble and radiation treatments result in a supra-additive effect in vivo. Endothelial cell apoptosis may be induced by ultrasound-stimulated microbubble treatments and enhanced with combined radiation treatments, leading to a reduction in tumor blood flow and inducing tumor cell death. By way of example, the target region may be exposed to radiation via an external beam radiation therapy source, a brachytherapy source, or a radioisotope therapy source. Exposure to the radiation dose may occur concurrently with the ultrasound exposure, or may occur some time period thereafter. For example, the target region may be exposed to a radiation dose anywhere from five minutes to twenty-four hours after sonification. The optimal interval time for synergistic interaction of ultrasound-stimulated microbubble exposure and radiation exposure is to provide the radiation exposure from about three hours to about twelve hours after the ultrasound-stimulated microbubble exposure for single fractions of combined treatments with the maximal synergistic effect occurring at about six hours. With the method of the present invention, traditionally non-curative doses of radiation combined with ultrasound-stimulated microbubble treatment can be made at least as effective as curative doses of radiation without ultrasound-stimulated microbubble ceramide accumulation. Other forms of energy may also be substituted for radiation.

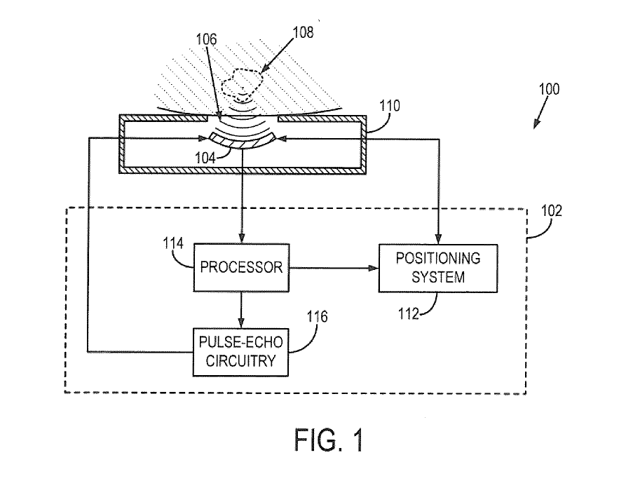

An example of an ultrasound system configured to generate an anti-angiogenic or anti-tumor bioeffect such as those described above may include an ultrasound transducer, a positioning system, a processor, and pulse-echo circuitry, which may include a waveform generator, a power amplifier with pulser/receiver, and a digital acquisition system. Referring now to FIG. 1, an example of an ultrasound system 100 configured to generate such an anti-angiogenic or anti-tumor bioeffect is illustrated. The ultrasound system 100 is controlled by an ultrasound control system 102. The ultrasound system 100 includes an ultrasound transducer 104 that is configured to transmit an ultrasound beam 106 to a target region 108 in a patient. By way of example, the ultrasound transducer 104 may be capable of producing a focused ultrasound beam.

The ultrasound transducer 104 is preferably placed in direct or nearly direct contact with the subject. In some configurations, the ultrasound transducer 104 may be housed in an enclosure 110 to provide an interface with the patient such that the ultrasound beam 106 can be efficiently transferred from the ultrasound transducer 104 to the target region. By way of example, the enclosure 110 may be filled with an acoustic coupling medium, which allows for a more efficient propagation of ultrasound energy than through air. Exemplary acoustic coupling media include water, such as degassed water.

The top of the enclosure 110 may include a flexible membrane that is substantially transparent to ultrasound, such as a flexible membrane composed of Mylar, polyvinyl chloride (“PVC”), or other plastic materials. In addition, a fluid-filled bag (not shown) that can conform easily to the contours of a patient placed on the table may also be provided along the top of the patient table.

The ultrasound transducer 104 may be connected to a positioning system 112 that provides movement of the transducer 104 within the enclosure 110, and consequently mechanically adjusts the focal zone of the transducer 104. For example, the positioning system 112 may be configured to move the transducer 104 within the enclosure 110 in any one of three orthogonal directions, and to pivot the transducer 104 about a fixed point within the enclosure 110 to change the angle of the transducer 104 with respect to a horizontal plane.

The ultrasound controller 102 generally includes the positioning system 112, a processor 114, and pulse-echo circuitry 116. The pulse-echo circuitry 116 is configured to provide a driving signal that directs the ultrasound transducer 104 to generate the ultrasound beam 106. For example, the pulse-echo circuitry 116 receives control parameters, such as pulse repetition frequency, peak negative pressure, exposure duration, and center frequency from the processor 114 and uses these control parameters to produce an effective ultrasound exposure. The processor 114 is also in communication with the positioning system 112, and is configured to direct the positioning system 112 to move the position of the ultrasound transducer 104 so that the ultrasound beam 106 will be transmitted to the target region 108.

A system and method for generating an ultrasound-induced, anti-angiogenic bioeffect in which key genes involved in ceramide induced apoptotic pathways are activated after ultrasound exposure in the treatment with microbubbles have been provided. The system and method of the present invention achieve therapeutic ceramide accumulation in targeted cells, thereby reducing the radioresistance of those cells or otherwise inducing apoptosis or other cellular death.

Microbubble-activated ultrasound treatments could be focused in an image-guided manner to just the tumor alone, as is already done with high-power thermal treatments, minimizing normal tissue toxicity. Further, there could be a differential sensitivity in normal tissues as tumor microvasculature is functionally abnormal. Additionally, such combined treatments could be used to decrease the total dose of radiation, which would further mitigate normal tissue radiation treatment-limiting toxicities. Also, these vascular disrupting ultrasound-activated microbubble treatments could be added to stereotactic high-precision radiation treatments to take advantage of vascular responses.

Having described systems and methods that implement the present invention, generally, several non-limiting examples of the present invention in use are now provided.

### EXAMPLE 1

### Tumor Radiation Response Enhancement by Acoustical Stimulation of the Vasculature

In this example, the inventors demonstrate that low mechanical index ultrasound-mediated excitation of microbubbles can enhance the effects of radiation in vitro and supra-additively in vivo using histological and functional assays of cell death and tumor growth delay experiments.

Data obtained from experiments in vitro indicate that, under these ultrasound-exposure conditions, ceramide formation is induced by microbubble interactions with cells and is also associated with endothelial cell apoptosis. Endothelial cell death in vivo caused by microbubble perturbation of tumor microvasculature leads to a pronounced vascular disruption and a 10-fold enhancement of tumor cell death when combined with single radiation treatments. Experiments indicate that single 2-Gy doses of radiation can lead to more than 40 percent tumor volume kill. Treatments with multiple fractions of the combined modalities demonstrate that ineffective doses of radiation can be made more effective in terms of tumor growth delay and mouse survival.

### Materials and Methods

Human PC3 prostate cancer (ATCC) xenografts were grown in the hind upper leg of SCID-17 mice (Charles River) by injecting 1.0×10̂6 RPMI-1640 media cultured cells subcutaneously (Wisent Biocentre), with 10% characterized serum (HyClone), and 100 U/mL of penicillin/streptomycin (Invitrogen). Tumors were grown to 7-8 mm diameter size before treatment. For treatments, ketamine and xylazine anesthetized mice were used. Vialmix device-prepared DEFINITY® (Lantheus Medical Imaging; N. Billerica, Mass.) microbubbles (perfluoropropane gas/liposome shell) were administered at doses of 3.6×10̂8 microbubbles (L, low dose) and 1.08×10̂9 microbubbles (H, high dose) in 30-4 and 90-4 volumes of prepared bubbles, respectively. The final circulating concentrations were selected to be higher (100- and 300-fold, respectively) than the diagnostic dose used to ensure efficient interactions of the bubbles and microvascular walls.

Mice were immersed in a 37 degree Celsius water bath to permit ultrasound treatment and centered on the tumor. For ultrasound exposures, a focused central frequency 500-kHz transducer (IL0509HP; ValpeyFisher Inc.) with a 28.6-mm transducer element diameter was used. This was attached to a micropositioning system, and excited with sinusoidal wave generated by a waveform generator (AWG520; Tektronix), a pulse-receive power amplifier (RPR4000; Ritec Inc.), and a digital-acquisition system (Acquiris CC103, Agiulent Technologies NY). Tumors were exposed over 50 ms to a 16-cycle tone burst at 500-kHz and 3-kHz pulse repetition frequencies with a 10% duty cycle during the 50-ms window. Treatments were for 5 min, amounting to a 750-ms exposure over 5 min for all mouse treatments, with an average duty cycle of 0.25%. Specifically, at 500 kHz the pulse bandwidth of the 16-cycle tone burst was 0.032 ms. The pulse repetition period (3-KHz pulse repetition frequency) was 0.333,ms, which, over 50 ms, corresponded to 150 periods of 16-cycle tone burst or 4.8 ms (rounded to 5 ms). This 5-ms time occurred every 2 s to permit blood vessels to refill with bubbles during a treatment time of 5 min (300 s), or 150 times, for a total time of 750 ms. The ultrasound peak negative pressure was 570 kPa measured with a calibrated hydrophone. The −6 dB beamwidth was 31 mm and the −3 dB beamwidth was 18 mm.

Immediately after ultrasound exposure, mice were lead-shielded and only tumor was exposed to ionizing radiation (Faxitron Cabinet X Ray; Faxitron X Ray LLC) at doses of 0, 2, or 8 Gy in single fractions using a dose rate of 200 cGy/min.

Mice were kept for 24 hours and then killed for histopathology, and a portion used for clonogenic survival assays. A second cohort of mice was used for 30-d long-term survival and growth delay analysis. A third cohort was used for micro-ultrasound power Doppler imaging. Each cohort had six mice per condition with 54 mice per cohort. Mice were also exposed to ultrasound alone and microbubbles alone as controls.

### Single-Fraction Experiments

We tested the hypothesis that combined mechanical disruption of endothelial cells and radiation can result in synergistic tumor cell kill in vivo. For ultrasound treatments, PC3 prostate cancer xenograft-bearing mice were administered microbubbles intravenously, which were stimulated using ultrasound to cause endothelial cell perturbations only within tumor vasculature. Three sets of mice (n=36×3), in addition to controls, were used to investigate acute effects, longitudinal effects, and blood flow non-invasively.

Experimental conditions included no microbubbles, a low, and a high concentration of microbubbles activated by ultrasound. Each of these was coupled with 0, 2 or 8 Gy of radiation given in one fraction resulting in nine experimental conditions with four mice per group (n=36). Mice were treated intentionally with combined single fractions to investigate combined effects. Other control conditions including ultrasound exposure in the absence of bubbles, and bubble injections without ultrasound, were investigated.

In order to ensure that microbubbles replenished the microvasculature between pulses designed to cause microbubble disruption, the ultrasound pulse sequence for mouse treatments was transmitted using a 10 percent duty cycle within a 50 millisecond window every 2 seconds for a total active insonification time of 750 milliseconds over 5 minutes for an overall duty cycle of 0.25 percent. Microbubble disruption was carried out at a diagnostic ultrasound exposure range using a pressure of 570 kPa for a mechanical index of 0.76. These parameters were chosen to prevent tissue heating and thermal damage, which are theoretically negligible at these conditions.

The first set of mice was sacrificed for histological analysis 24 hours after treatment. This time was selected to maximize potential tumor cell death secondary to gross vascular disruption due to endothelial cell apoptosis.

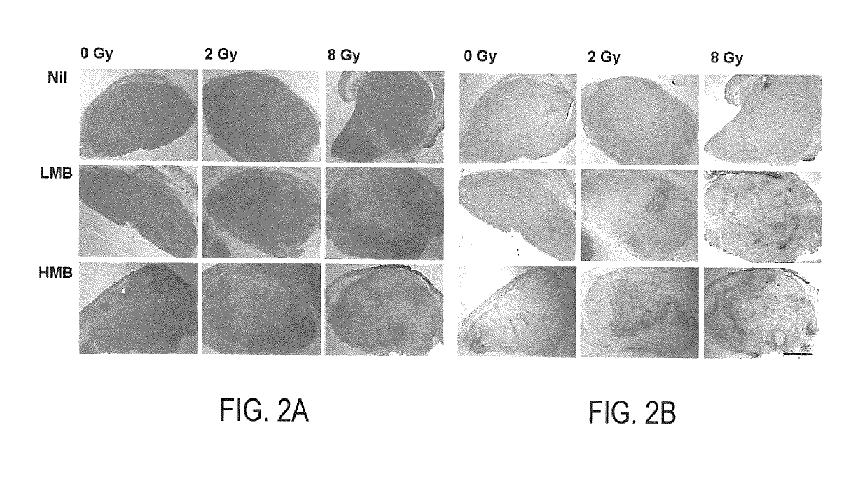

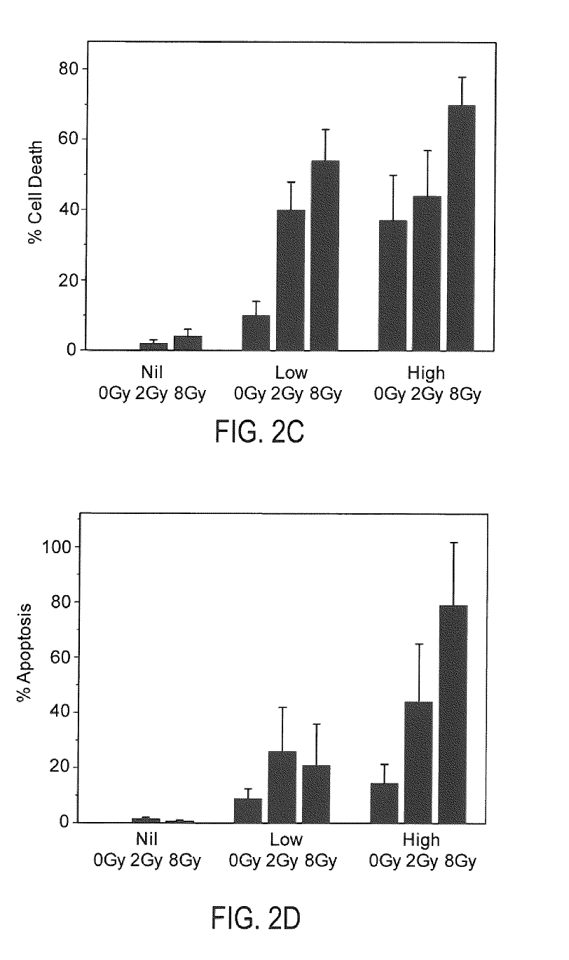

Results indicated that the combination of ultrasound-stimulated microbubble treatment with radiation resulted in a significant induction of cell death. Representative data presented in FIGS. 2A and 2B indicate extensive increases in cell death with combined treatments. Treatment with 0 Gy, or a single 2 Gy or 8 Gy fraction of radiation alone resulted in minimal apoptotic or necrotic cell death (4±2% death for 2 Gy, mean±SE) as did ultrasound-activated microbubble treatment alone (10±4% death for the low bubble concentration). In contrast, the combination of these resulted in macroscopic regions of apoptotic and necrotic cell death in the area of ultrasound microbubble activation occupying approximately 40±8% or more of the tumor cross-sectional area for the 2 Gy dose combined with the low-microbubble treatment with ultrasound. The combined 2 Gy and high microbubble concentration resulted in more cell death (44±13%) and the combination of 8 Gy and the high microbubble concentration resulted in even more cell death (70±8%).

Quantification of tumor cell death indicated a supra-additive effect between radiation and the ultrasound treatments (FIG. 2C) with increasing apoptosis observed with the combined treatments. Quantitative analysis of histopathology results confirmed a supra-additive effect between the ultrasound-activated microbubble treatments and the effect of radiation. There were non-linear increases in macroscopic measurements of cell death evident when the ultrasound-activated microbubble treatments were combined with radiation treatments. The combination of the two treatments also led to non-linear increases in the number of apoptotic cells (FIG. 2D).

Additional control treatments with ultrasound alone, and with injected microbubbles in the absence of ultrasound, demonstrated no significant difference in comparison to untreated mice (P<0.05). Statistical analysis by two-way ANOVA indicated a significant effect of radiation (P=0.0002), effect of microbubble treatments (P<0.0001), and indicated an interaction between radiation and microbubble treatment (P<0.0002). The combined 2 Gy and microbubble treatments were significantly different compared to 2 Gy alone or ultrasound-activated microbubble treatments alone, for the ultrasound-activated low and high-microbubble concentration treatments, respectively (all P values<0.0001). This was also observed for the combined 8 Gy and microbubbles treatments compared to 8 Gy, or ultrasound-microbubble treatments alone (all P values<0.0001).

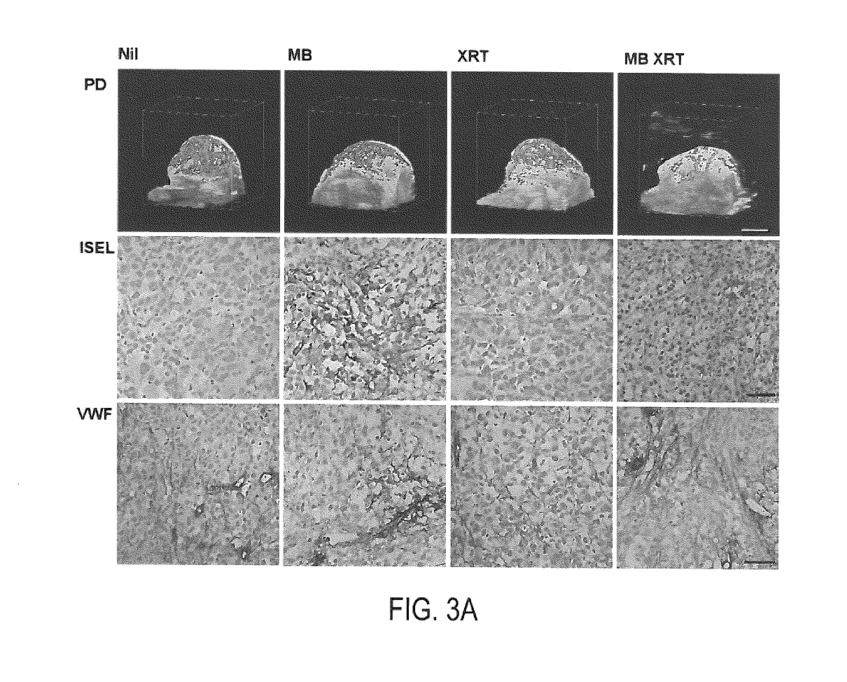

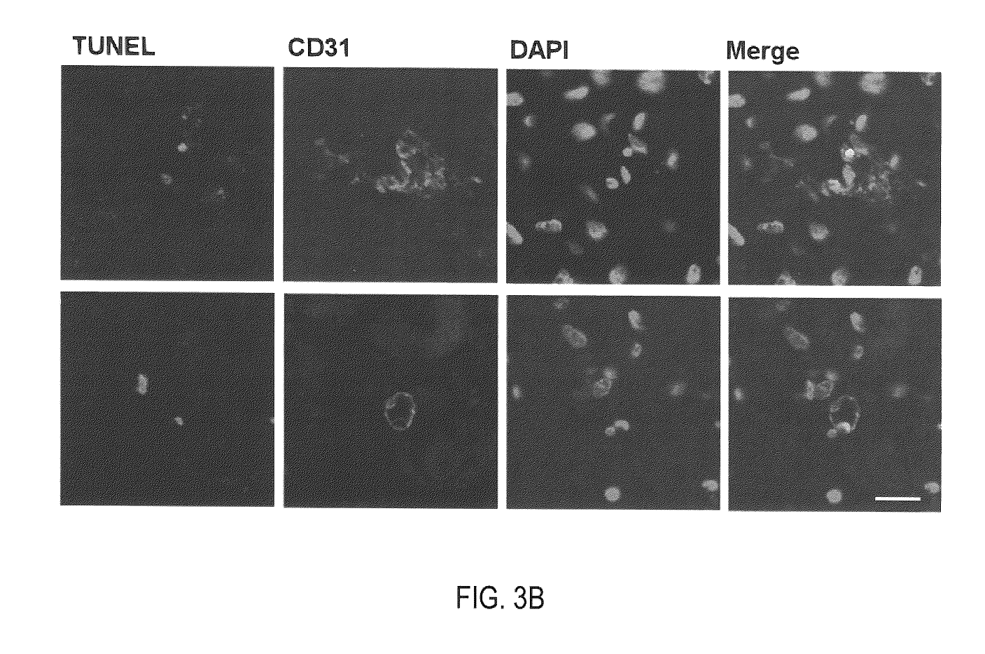

In order to investigate the mechanism behind this enhancement of cell death we utilized non-invasive imaging techniques to track effects on the vasculature as well as immunohistochemical histology methods. Power Doppler micro-ultrasound imaging was carried out in a separate cohort of mice under the same experimental conditions (n=36). Doppler data demonstrated moderate vascular disruption with ultrasound and microbubbles, and with 8 Gy radiation doses (20±21% and 20±32% decrease in Doppler vascular-index, respectively). Significant reductions in blood flow at 24 hours for the combined ultrasound-activated microbubble and radiation treatments were observed suggestive of vascular disruption (65±8% decrease in Doppler vascular-index). The combination with ultrasound-stimulated microbubbles and radiation was significantly better in flow diminishment compared to the single treatments (P<0.001) (FIG. 3A). The effect of the combination treatments was more consistent with a smaller standard error compared to individual treatments. Corresponding immunohistochemistry under high-power microscopy indicated that ultrasound-activated microbubble treatments resulted in microscopic localized appearances consistent with endothelial cell apoptosis, whereas combined ultrasound-activated microbubble and radiation treatments resulted in near total cell death of endothelial cells and tumor cells that was not apparent at the other experimental conditions (FIG. 3A).

Analysis (ANOVA) indicated that ISEL staining levels for ultrasound-stimulated microbubble treatment in combination with radiation (70±8%) were significantly different in comparison to radiation alone (4±2%) or ultrasound-stimulated microbubble exposure alone (36±12%) (both P<0.001) 24 hours after treatment for the higher microbubble concentration.

Immunohistochemical staining of von Willebrand factor revealed enhanced leakage from the vasculature with the combined ultrasound-activated microbubble and radiation treatments further suggestive of vascular disruption (FIG. 3A). In order to investigate the mode of endothelial cell death being induced by the ultrasound treatments in the presence of microbubbles, confocal microscopy of triple-immunohistochemical stained sections of ultrasound-activated microbubble treated xenograft tumors sections confirmed the induction of apoptosis in endothelial cells in tumors treated with ultrasound and microbubbles (FIG. 3B). Analysis (ANOVA) indicated that staining levels for ultrasound-stimulated microbubble treatment (low bubble concentration) in combination with radiation (8±1%) were significantly different in comparison to radiation alone (2±1%) or ultrasound-stimulated microbubble exposure alone (6±1%) (both P<0.001) 24 hours after treatment. These values were for the low-microbubble concentration and were consistent in general with ISEL staining of whole tumor at that concentration of microbubbles: 18±15% apoptosis for radiation and ultrasound and microbubbles, 1±4% for radiation alone, and 7±4% apoptotic-index for ultrasound-stimulated microbubble exposure alone (FIG. 2C).

### Targeted-Microbubble Experiments and bFGF Experiments

The effect of microbubble proximity to endothelial cells in the observed radiation-enhancing effect experiments with non-targeted and vascular endothelial growth factor receptor (“VEGFR2”)-targeted microbubbles was investigated and indicated increases in cell death with the targeted microbubbles (P=0.005). For targeted experiments, avidin-conjugated MicroMarker Target-Ready Agent (VisualSonics) was used with biotinylated VEGFR2 antibody (Abcam) with a bubble concentration equivalent to that for the low concentration DEFINITY® experiments. Unconjugated and conjugated bubbles were used for experiments with ultrasound parameters as described previously for experiments with DEFINITY® microbubbles with four PC3-bearing mice per group.

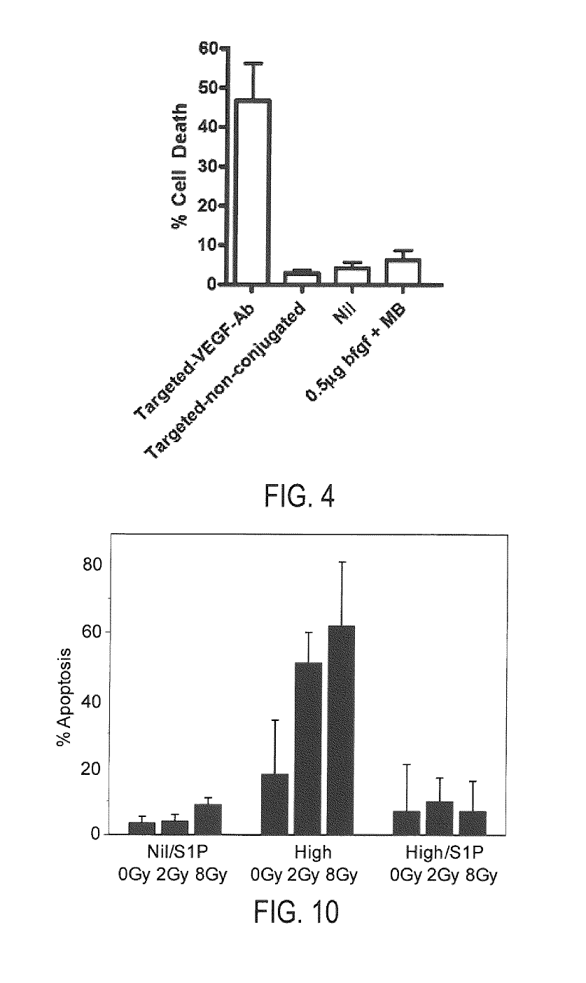

In order to test further the importance of disrupting endothelial cells, PC3-bearingmice were treated with 0.45 μg basic fibroblast growth factor (“bFGF”) IV (Sigma), a known endothelial cell protector, 1 hour prior to exposure to microbubbles alone (1% vol/vol) in the presence of ultrasound stimulation (n=5). For non-targeted treatments, pre-treatment of animals with basic fibroblast growth factor (“bFGF”) diminished the cell death that microbubble treatments induced. In addition, there was no difference between bFGF-treated animals when treated by ultrasound-stimulated microbubbles and untreated control in terms of cell death (P=0.24) (FIG. 4).

### Exposure Experiments

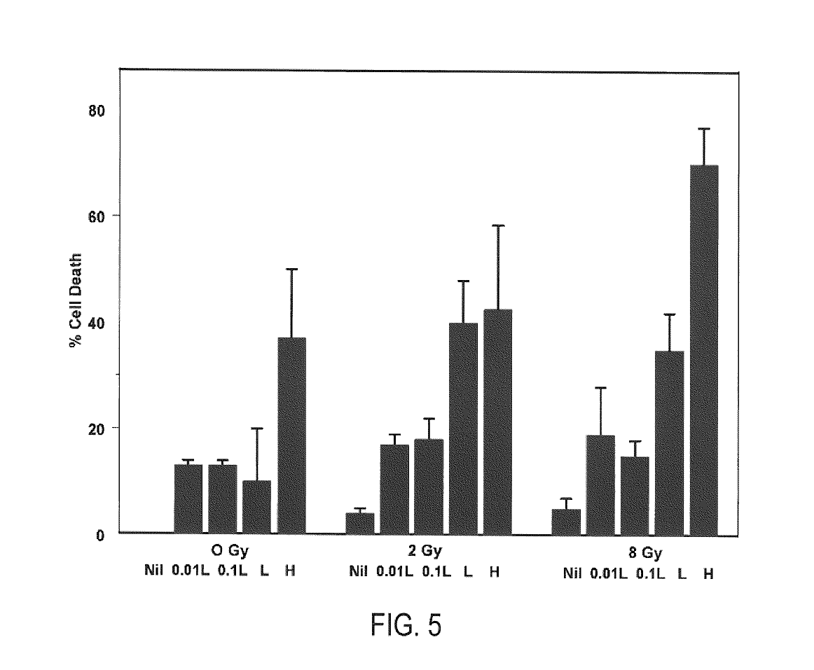

In order to investigate the effect of ultrasound-stimulated microbubble exposure, experiments were conducted in which the concentration of microbubbles was varied (FIG. 5). For experiments the microbubble concentration was varied to include zero, to 0.01 times the low concentration, 0.1 times the low concentration, the low concentration, and the high concentration. These concentrations were combined with 0, 2, and 8 Gy radiation dose treatments. Statistical analysis using ANOVA indicated an interaction accounting for 11% of the total observed effect (P<0.001). Analysis with ANOVA indicated radiation dose accounted for 10% of the effect (P<0.001) and microbubble dose accounted for 70% of the observed effect (P<0.001) (n=4 for all groups). Treatment effects were present at 0.01 of the low concentration (approximate clinical imaging concentration) of microbubbles but increased at the higher concentrations. With the 2 Gy doses exposure to the low and high microbubble concentrations produced equivalent results with better results at the higher concentration of bubble combined with 8 Gy radiation treatment.

### Timing Experiments

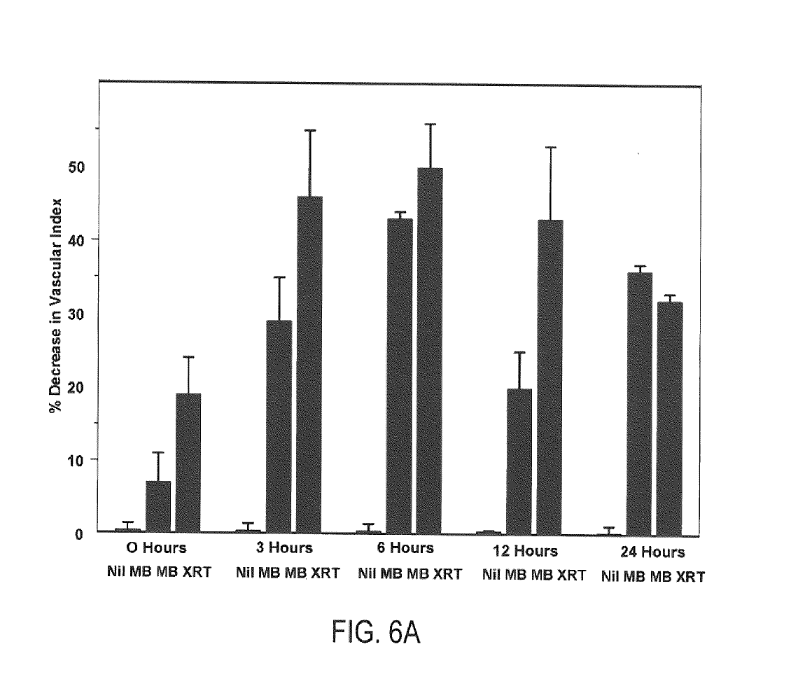

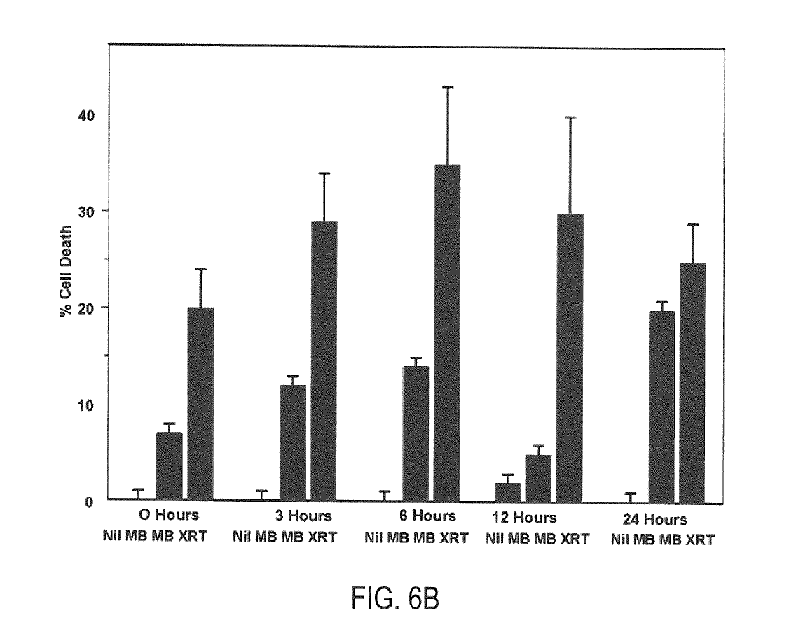

Effects of ultrasound-stimulated microbubble exposure and resultant effects on cell death and micro-Doppler-detected blood flow were investigated. This modality was investigated alone and with a sequence of a time delay introduced with subsequent radiation treatment (0, 3, 6, 12, and 24 hours) (n=4 for all groups). Treatment with ultrasound-stimulated microbubbles indicated maximal cell death detected using ISEL staining when the two treatments were separated by 6 hours, which coincided with a maximal decrease in detected micro-Doppler blood-flow signal. Radiation at that time resulted in a maximal effect 24 hours later, in terms of ISEL detected cell death and disruption of blood flow-linked micro-Doppler detected signal (FIGS. 6A and 6B). The data imply a 9 hour window for radiation therapy after microbubble exposure with no statistically significant difference between results from 3 to 12 hours.

For time-interval experiments ANOVA indicated a statistically significant radiation effect (P<0.0001), a statistically significant microbubble effect (P<0.0001) and an interaction between the two treatments (P<0.0001) for cell death and blood flow-disruption each.

### Single-Fraction Growth Delay

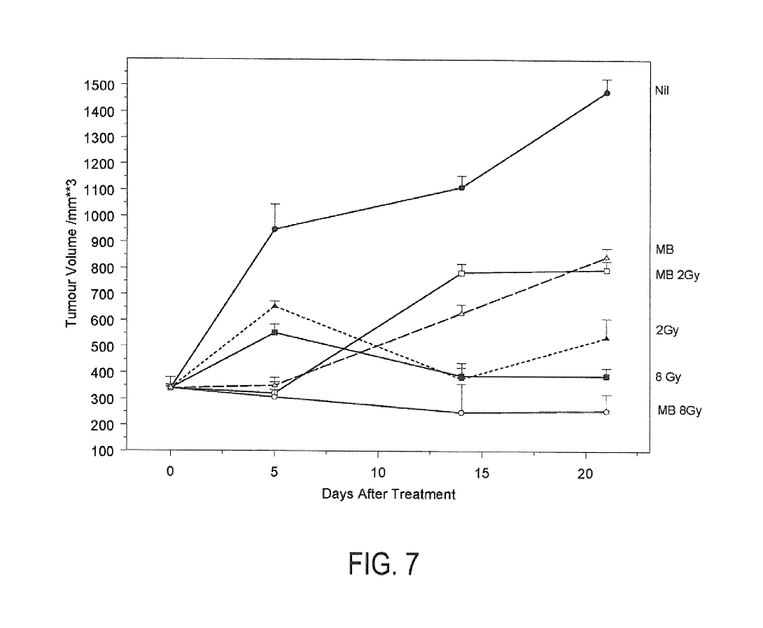

Another cohort of mice (n=36) was treated with the same nine conditions used initially for single treatments, except that mice were followed longitudinally after treatments for growth delay effects of single treatments, bearing in mind that ultrasound and microbubble treatments as delivered resulted in a viable tumor rim. At 5 days, combined ultrasound-activated microbubble and 2 Gy or 8 Gy radiation treatments yielded the greatest growth delay (FIG. 7). Ultrasound-activated microbubble treatments yielded a similar delay in xenograft tumor growth. Radiation treatments alone were less effective at arresting tumor growth with tumor growth still evident at 5 days duration. At 20 days after treatment with ultrasound-activated microbubble treatments alone or combined with 2 Gy there was rebound growth of tumor xenografts. The combined ultrasound-activated microbubble treatment and 8 Gy radiation treatment effectively inhibited tumor growth whereas 8 Gy alone began to show regrowth at 21 days.

Growth 5 days after combined treatment with 2 Gy radiation and ultrasound-stimulated microbubbles was significantly different from 2 Gy alone (P<0.01) but not 8 Gy. Ultrasound-stimulated microbubble treatment alone was different compared to 2 Gy or 8 Gy alone (P<0.01). At day 20 after growth rebound there was no difference in growth delay between 2 Gy combined with ultrasound-stimulated microbubbles compared to 2 Gy alone. Ultrasound-stimulated microbubble treatment alone was not different compared to 2 Gy and had less growth delay than 8 Gy alone (P<0.01). There was no statistically significant difference in growth delay between ultrasound-stimulated treatment with 2 Gy in comparison to 8 Gy (P<0.05).

### Multiple Fraction Growth Delay

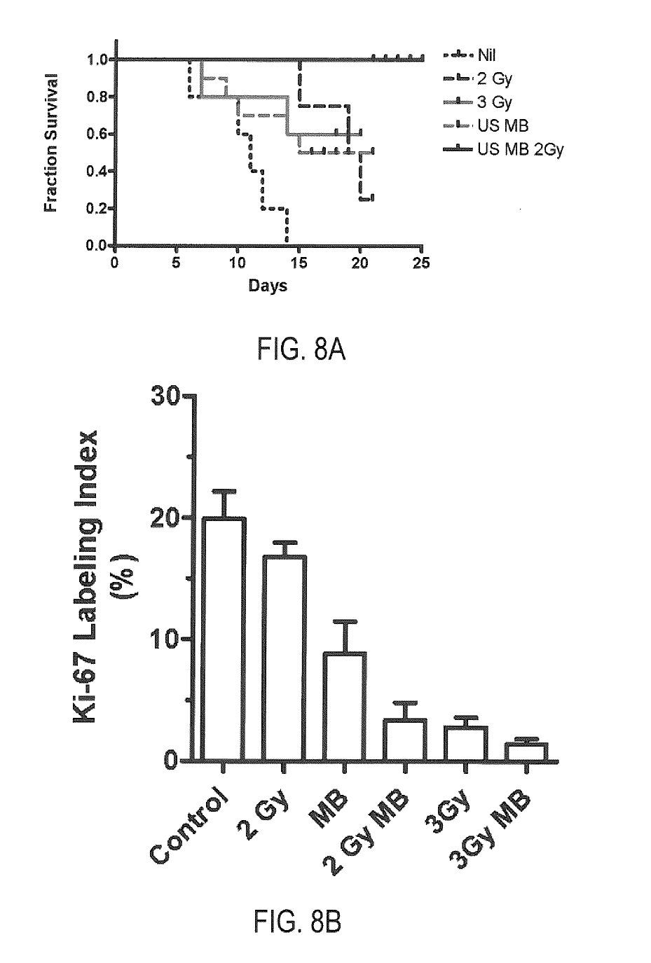

Analysis of associated growth and survival curves and Ki-67 activity (as a marker of cellular proliferation) is presented in FIGS. 8A and 8B. Analysis of survival curves to mouse death or modified human endpoint or 2 cm tumor size indicated that they were significantly different (P<0.05) with mean survivals of 10±1, 19±1, 20±3, 25±3, and 28±0 days (mean±standard error) for mice receiving no treatment, and treatment with the 2 Gy fractionation scheme (BED(10)=28.8 Gy), the ultrasound-stimulated microbubble regimen, the 3 Gy fractionation scheme (BED(10)=58.5 Gy), and the combined ultrasound-stimulated microbubble and 2 Gy radiation fractionation regimen. Growth delay data indicated there was no significant difference between 2 Gy combined with ultrasound-stimulated microbubbles compared to the 3 Gy regimen.

For analyses of growth delay at day 21, the 2 Gy regimen combined with ultrasound-stimulated microbubbles was significantly different from 2 Gy alone (P=0.03) and significantly different from the ultrasound treatments alone (P=0.02). There was no significant difference in terms of growth delay at day 21 between the 2 Gy regimen combined with ultrasound-stimulated microbubbles and the 3 Gy regimen. Adding ultrasound stimulated-microbubbles to the 3 Gy regimen made no statistically significant difference in terms of growth delay. Analyses results using the non-parametric Mann-Whittney analysis were equivalent.

Further analysis using Ki-67 labeling (FIG. 8B) indicated that the ultrasound-stimulated microbubble treatment in combination with ultrasound was significantly different in comparison to the 2 Gy regimen alone (P<0.001). There was no statistically significant difference between this combined regimen and the 3 Gy radiation alone regimen (P>0.05). Analysis using ANOVA indicated (P<0.007) an interaction between radiation and microbubble treatment.

### Ceramide and Sphingosine-1-Phosphate Experiments

In order to test if ultrasound-stimulated microbubbles in combination with ultrasound could stimulate ceramide formation, experiments were carried out using an additional cohort of 75 animals, with five mice per group. Mice were treated as above with no microbubbles, low, and high bubble concentrations in the presence of ultrasound and combined with 0, 2, and 8 Gy radiation doses given in single fractions as above for nine cohorts of n=5 animals. In addition, 0, 2, and 8 Gy conditions with and without high-concentration microbubble exposure in the presence of ultrasound were carried out in the presence of S1P using modified protocols. This used six cohorts of n=5 mice.

For S1P treatments, 4 μg of S1P in 0.2 mL of PET (5% polyethylene glycol, 2.5% ethanol, and 0.8% Tween-80) was injected intravenously in mice 30 min prior to and 5 min after irradiation or after microbubble exposure with ultrasound and irradiation.

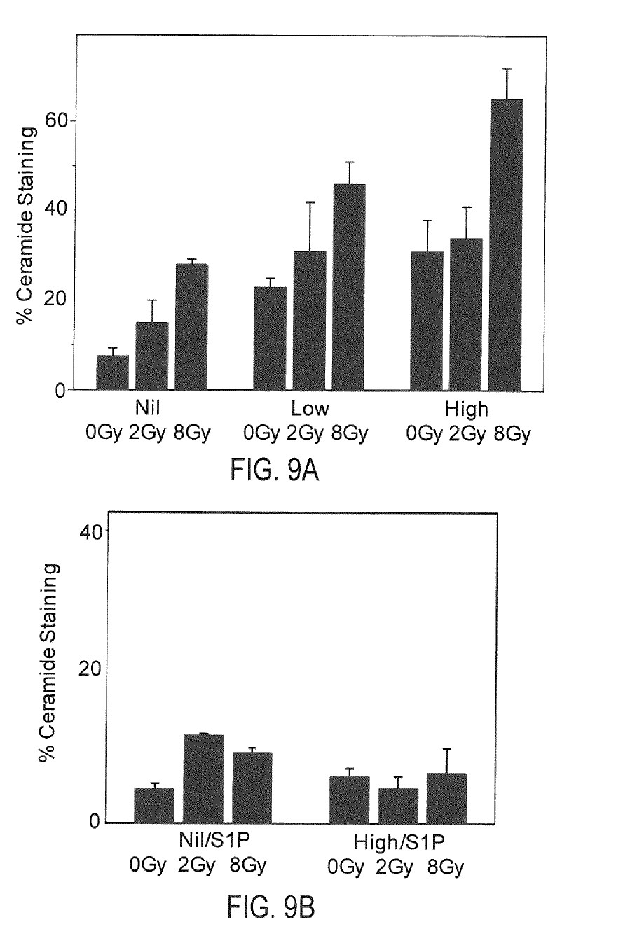

Analyses of experiments from this additional cohort of 75 animals (n=5 mice per group) indicated increases in ceramide formation in vivo with microbubble exposure combined with radiation exposure (FIG. 9A). Statistical analysis using ANOVA indicated that bubble dose accounted for 32% of the total effect (P=0.0003). Radiation dose accounted for 32% of the effect (P=0.0004). Compared to no treatment, the combination of either the low concentration or the high concentration of microbubbles with 8 Gy resulted in significant ceramide staining (P<0.05, P<0.001, respectively). This was not as apparent for treatments using ultrasound-stimulated bubbles alone or radiation alone. Treatment with 8 Gy alone resulted in ceramide increases that were readily apparent, but not significant. In the presence of sphingosine-1-phosphate (“S1P”), which inhibits ceramide synthesis, increases were not apparent (FIG. 9B). Statistical analysis indicated no significant increases in ceramide for the 2 Gy or 8 Gy dose in the presence of the high-microbubble concentration.

Corresponding analyses of cell death are presented in FIG. 10. Data indicate apoptotic cell death was induced by ultrasound-stimulated microbubbles when combined with radiation but inhibited by S1P exposure. Treatment with S1P, given 30 minutes before and 5 minutes after treatments, resulted in a diminishment of detected apoptotic cell death with no statistically significant difference between 0, 2, and 8 Gy treatments in the presence of S1P (as control) and ultrasound-stimulated microbubble-exposure combined with 0, 2, and 8 Gy treatments in the presence of S1P. In the absence of S1P, ultrasound-stimulated microbubble exposure and 0, 2, and 8 Gy treatments exhibited statistically significant levels of cell death as before. Apoptotic cell morphology and ISEL staining was diminished in the presence of SIP.

Treatments involving multiple fractions of radiation combined with ultrasound and microbubbles demonstrated a greater therapeutic effect compared to radiation alone. Non-curative doses of radiation combined with ultrasound-stimulated microbubble treatment were at least as effective as curative doses of radiation. Results obtained in vivo with S1P as a ceramide cell death pathway inhibitor demonstrate that ceramide accumulation is involved in responses to ultrasound microbubble treatments.

Potential cellular mechanisms other than activation of the ceramide pathway that result in the therapeutic effect described here include mechanical perturbation leading directly to endothelial cell apoptosis, cytokine stimulation, and changes in ionic environment caused by vascular disruption. Microbubbles and ultrasound may also cause biochemical reactions when depositing energy near cell membranes, leading to lipid reactions. However, the increases in ceramide production caused by microbubbles, particularly when combined with radiation, seemed to suggest activation of stress related lipid metabolism, likely in response to cell membrane damage.

The experiments conducted here were mainly carried out using microbubble concentrations at 100-300 times greater than those used diagnostically, but also used concentrations at the diagnostic dose of microbubbles. These lower concentrations exhibited a lesser effect when combined with radiation; however, these treatments were carried out with an overall duty cycle of 0.25% (750 ms insonifcation over 5 min). In order to compensate for lower microbubble concentrations, exposure to microbubble oscillations could be increased by increasing the duty cycle, because it is contemplated that the ceramide accumulation and cell death-inducing effect is related to the number of bubbles insonified.

Combined ultrasound and radiation treatments were demonstrated to improve the effects of radiotherapy, which is commonly given in multiple-fraction treatments. This could be envisaged as a conformal method of enhancing radiation responses. Microbubble-activated ultrasound treatments could be focused in an image-guided manner to just the tumor alone, as is already done with high-power thermal treatments, minimizing normal tissue toxicity. Further, there could be a differential sensitivity in normal tissues as tumor microvasculature is functionally abnormal. Additionally, such combined treatments could be used to decrease the total dose of radiation, which would further mitigate normal tissue radiation treatment-limiting toxicities. Lastly, these vascular disrupting ultrasound-activated microbubble treatments could be added to stereotactic high-precision radiation treatments to take advantage of vascular responses.

### EXAMPLE 2

### Ultrasound-Activated Microbubble Cancer Therapy: Ceramide Production Leading to Enhanced Radiation Effect In Vitro

In this example, the inventors demonstrate with proof-of-principle experiments that ultrasound and microbubbles can be used to additively enhance radiation effects. The inventors also demonstrate this method results in an accumulation of ceramide in endothelial, leukemia, breast cancer, prostate cancer, and murine fibrosarcoma cells. The inventors further test the importance of the asmase pathway using asmase +/+ and asmase −/− astrocytes in addition to drug inhibition of the ceramide pathway.

Because microbubbles administered intravenously are likely to damage only vascular endothelial cells, the choice was made to use HUVEC cells for this study. Astrocytes were chosen also as a test system for their relative radioresistance. Astrocytes also represent a good target for new ultrasound-stimulated interstitial microbubbles and permitted the investigation of the asmase pathway as stable cultures were available from wild type and asmase knock-out mice.

The inventors demonstrate that microbubbles have the potential to maximize the effects of radiation by inducing the synthesis of pro-apoptotic intracellular ceramide. This technique may thus be used as a radiation enhancer to achieve greater tumor eradication and avoid the use of higher doses of radiation.

### Materials and Methods

### Cell Cultures

All cells were grown at 37 degrees Celsius with 5% CO₂. Primary astrocytes (obtained from asmase +/+ and asmase −/− mouse brains, Sunnybrook Health Sciences Centre, Toronto, ON) were cultured in DMEM with 10% fetal bovine serum (FBS) and 5% Penicillin. HUVEC cells (Sunnybrook Health Sciences Centre, Toronto, ON, Canada) were grown in EBM-2 (Lonza, Walkersville, Md. USA) supplemented with 10 ml FBS, 0.2 ml Hydrocortisone, 2 ml hFGF-B, 0.5 ml VEGF, 0.5 ml R3-IGF-1, 0.5 ml ascorbic acid, 0.5 ml hEGF, 0.5 ml GA-1000 and 0.5 ml Heparin using EGM-2 singlequots kits. Breast and PC3 cells were grown in 1640-RPMI medium (Sigma-Aldrich Canada Inc., Oakville, ON, Canada), leukemia (AML, Ontario Cancer Institute, Toronto, ON), and KHT sarcoma cells (Sunnybrook Health Science Centre) were grown in α-MEM (Sigma-Aldrich Canada Inc., Oakville, ON), all with 10% FBS and 5% penicillin. In order to harvest adherent cells, confluent flasks were PBS washed, after which 0.05% trypsin EDTA (Gibco, Carlsbad, Calif. USA) was added for 5 minutes to detach cells. After trypsinization, cells were centrifuged at 440 g for 10 minutes and brought to a final cell concentration of 2×10̂6 cells/ml of medium with 1.5 ml aliquots used for each treatment (i.e., 3×10̂6 cells/sample). In all experiments cells were carefully handled to avoid clumping after typrinsization and this was verified by microscopy in advance of ultrasound treatments.

### Treatments

For ultrasound treatments, a pulse was generated by a 2.86 cm-diameter single element transducer (IL0509HP, 500 kHz center frequency, Valpey-Fisher Inc., Hopkinton, Mass., USA) connected to a micro-positioning system. The set-up also included a cylindrical chamber (10 mm diameter) for cell exposure. The chamber had mylar windows on both sides and a magnetic stirrer to mix cells and bubbles during ultrasound exposure in order to avoid standing wave effects.

In order to treat the cells, 50 μL of 45 seconds agitated vial-mix DEFINITY® microbubbles (Perflutren lipid microspheres, Lantheus Medical Imaging, Billerica, Mass. USA) and 1.5 ml of the 2×10̂6 cells/ml solution were added to the chamber (for a 3.3% v/v bubbles concentration). Insonification took place using a peak negative pressure of 570 kPa using a pulse sequence with a 9.6% duty cycle composed of a 16 cycle tone burst and a pulse repetition frequency of 3 kHz, for a total insonification time of 2,880 milliseconds over 30 seconds. The −6 dB beamwidth for this transducer was 31 mm and the −3 dB beamwidth was 18 mm. Transducer characteristics were measured using a calibrated hydrophone in the absence and presence of the treatment set-up.

Sphingosine-1-Phosphate (S1P) (Biomol International L.P., Plymouth Meeting, Pa. USA) was used to counteract the mechanisms leading to ceramide-mediated apoptotic cell death. In order to treat cells, 1 μM S1P was added to asmase +/+ and asmase −/− culture media one hour before damage treatments with radiation or ultrasound, and was present during trypsinization, and for clonogenic survival assays in media. The rationale for this was that any activation of ceramide-dependent cell death by treatment may take many hours to manifest and should be inhibited long term. Treatment of cells with C2-Ceramide (Sigma-Aldrich) was carried out similarly.

Radiation treatments were carried out by exposing samples to X-ray ionizing radiation (Faxitron Cabinet X-ray, Faxitron X-Ray LLC, IL) at a dose rate of 200 cGy/minute. For combined treatments radiation treatments were given within 1-2 minutes of ultrasound exposure.

### Immunohistochemistry

Cells were added to generic cyto-spin cuvettes and cyto-spun onto Poly-L-lysine-(Sigma-Aldrich Canada Inc., Oakville, ON, Canada) coated slides at 1500 RPM for 2 minutes (Cytospin3, Shandon, Thermo Fisher Scientific Inc, Ottawa, ON, Canada). Cells were fixed with 4% paraformaldehyde (TAAB Laboratories Equipment Ltd, Aldermaston, England). Slides were subsequently immunostained for ceramide using a monoclonal anti-ceramide antibody (MID 15B4, Alexis Biochemicals, Plymouth Meeting, Pa. USA). Ceramide content was measured from immunohistochemistry by measuring the brown to blue ratio in cell microscopy images (Image J, NIH, Bethesda Md. USA). A brown to blue ratio of 8:2 was taken as positive for ceramide staining.

### Statistical Analysis

Survival assay were performed in duplicate, and within any one experiment, conditions were all done in triplicate (i.e. 3 culture dishes/sample and 2 samples per condition). Student's t-test were done to confirm statistical significance (Graph Pad Prism 4.0, La Jolla, Calif.)

### Results

### Cell Types and Ceramide Formation in Response to Microbubbles

Several cell types were exposed to microbubbles in the presence of ultrasound. Clonogenic assays indicated survivals of 12±2%, 65±5%, 83±2%, 58±4%, 58±3%, 18±7% for HUVEC, AML, PC3, MDA, KHT-C and asmase +/+ astrocyte cells. Histology and immunohistochemistry indicated that ceramide was formed in all cell types tested (AML, PC3, MBA231, KHT, HUVEC, astrocytes) in response to ultrasound-stimulated microbubble exposure. Immunohistochemistry using anti-ceramide antibody stained cells for ceramide after ultrasound exposure. This is specific antibody-medicated detection of ceramide with antibodies then stained using a colorimetric process.

Two cell types were selected for further analysis including HUVEC to test effects that may be involved in vivo upon endothelial cells subjected to intravascular exposure to ultrasound-stimulated microbubbles. Genetically modified astrocytes were also selected to probe the role of the asmase gene product. In addition, HUVEC cells were selected because they are highly enriched in asmase and sensitive to its activities, whereas astrocytes were selected because of low baseline levels of asmase and relative insensitivity to ceramide.

### Ultrasound-stimulated Microbubble Effects on Radiation-induced Cell Death in Endothelial Cells and Astrocytes.

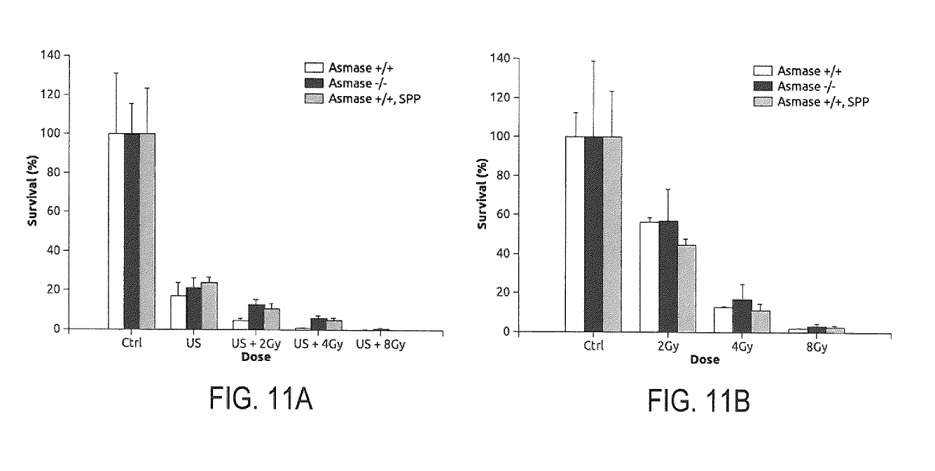

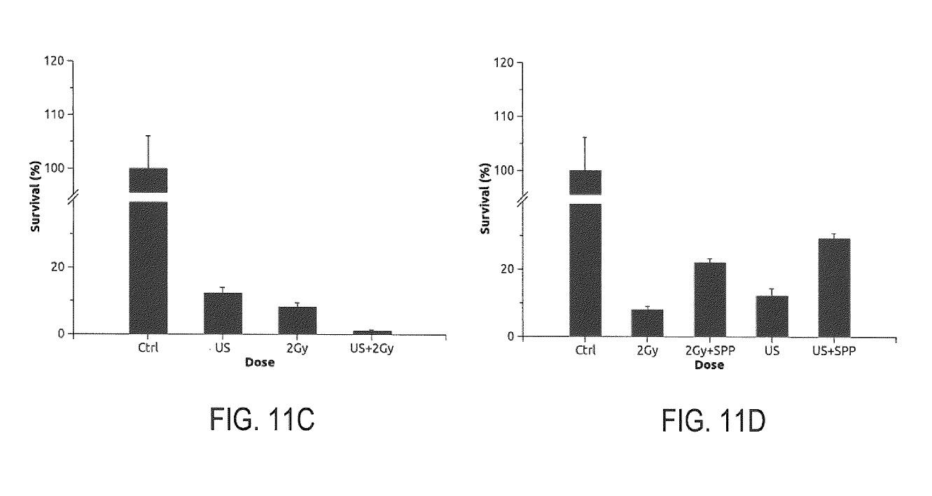

Survival assays of asmase +/+ and asmase −/− astrocytes, in response to ultrasound-activated microbubbles in combination with radiation (FIG. 11A), demonstrated a significantly enhanced level of cell death as compared to survival in response to radiation alone (FIG. 11B). The combination of the two treatments was additive. Survival decreased to 56±2% in response to 2 Gy radiation alone, whereas bubbles alone caused 17±7% survival. Only 5±2% survival or less in response to 2 Gy radiation with ultrasound-stimulated microbubble treatment was observed. A differential effect was also observed in the survival of asmase +/+ astrocytes and asmase −/− astrocytes in response to the combined treatments only (p<0.1 for the combination with 2Gy and p<0.05 for the combination with 4 Gy), whereas ultrasound and microbubbles treatment alone induced no observable differential effect (p>0.1).

Use of 1 μM S1P demonstrated no protective effect when asmase +/+ astrocytes were treated with either radiation or ultrasound with microbubbles (FIG. 11B), but did so when treated with the two modalities combined (FIG. 11A). Control astrocytes exhibited a 50±5% survival when treated with ceramide alone as a control. Survival assessment of HUVEC (FIG. 11C) revealed an additive effect when treated with ultrasound and microbbubles, combined with 2 Gy radiation. Here 1 μM S1P significantly protected the endothelial cells from apoptosis when the cells were treated with either radiation or ultrasound and microbubbles (p<0.01) (FIG. 11D).

### Ultrasound-Activated Microbubble Effects on Intracellular Levels of the Apoptotic 2^{nd} Messenger Ceramide.

Ceramide presence after treatment was assessed for HUVEC and astrocytes by immunostaining. Exposure of HUVEC cells indicated maximum detectable ceramide after treatment. For HUVEC cells treated with ultrasound-stimulated microbubbles and ultrasound-activated microbubbles with 8 Gy, 30-50% of cells exhibited ceramide staining. For astrocyte cells, little difference was observed amongst control, ultrasound-stimulated microbubble exposed and 8 Gy treated cells. In contrast, ultrasound-stimulated microbubble and radiation-exposed cells demonstrated a very high number of brown stained cells (70-80%). Asmase −/− astrocytes exhibited a lower survival response to bubble-induced ceramide compared to wild type astrocytes, but with similar 70-80% staining. We tested the effects of S1P to inhibit ceramide-related signaling in astrocytes as it was effective in doing so in HUVEC cells. Results indicated S1P conferred some radioresistance to wild-type astrocytes. Astrocytes treated with S1P and aSMase-deprived astrocytes had a higher and similar survival as compared to wild-type astrocytes.

Our results indicated that astrocytes did not exhibit apoptotic cell death with radiation treatment, which is consistent with previous studies (Li et al., Cancer Research 56(23):5417-5422 (1996)). In order to assess the inherent sensitivity of cells to ceramide, HUVEC and astrocytes cells were exposed to ceramide. Treatment of astrocytes with 1 μM c2-ceramide yielded no significant cell kill. HUVEC cells did not survive this exposure. The apoptotic blocker S1P failed to protect wild type astrocytes from cell death, indicating that these cells died from other types of cell death. Histologically, they exhibited mitotic arrest rather than apoptosis. However, S1P mediated protection was detected (FIG. 11A) when wild type astrocytes (+/+) were treated with ultrasound and microbubbles prior to radiation exposure. This suggests that ultrasound and microbubbles induce damage that causes wild type astrocytes to die by apoptosis.

Exposing different cell types (leukemia, HUVEC, fibro sarcoma, breast, prostate and astrocytes) to ultrasound and microbubbles indicated that ceramide formation in response to microbubbles-mediated cell membrane perturbation was present amongst different cells types. Astrocytes were selected to represent a relatively radiation resistant cell line. These cells could be treated in vivo with microbubbles by contact with liquid nano-droplets that can perfuse into tissue through leaky tumor vasculature where the nano-droplets can then be turned into gas bubbles by ultrasound exposure.

The experiments here were undertaken in order to characterize the potential for ceramide involvement in microbubble responses and to better understand the role of particular genetic pathways. No heating was detected in experiments and the power levels used were low, similar to those used in color Doppler.

Endothelial cell apoptosis is a key component of radiotherapy response. Its importance arises from the relationship between angiogenesis and tumor sustainment, given that endothelial cell death can lead to blood vessel collapse within a tumor. Recently, numerous studies have identified the apoptotic messenger ceramide as a useful tool to achieve higher endothelial cell apoptosis levels. Our data suggests that ultrasound-stimulated microbubbles in combination with radiation could be used as a radioenhancing modality. Data indicate that ultrasound-activated micro bubble exposure causes sufficient ceramide production to cause cell death. It also sensitized a relatively radioresistant cell type. The bioeffect elicited here also represents an excellent alternative to increasing radiation doses to improve cancer therapies, which often is not possible, and thus could enhance treatment effects at clinically relevant radiation doses.

### EXAMPLE 3

### Microbubble and Ultrasound Radioenhancement of Bladder Cancer

In this example, the inventors demonstrate that ultrasound-mediated microbubble treatment combined with radiation is useful in destabilising tumor vasculature and enhancing radiation response in bladder cancer xenografts in vivo.

### Materials and Methods

### Cell Culture

Cell lines were obtained from the American Type Culture Collection (ATCC, Manassas Va.). Human bladder carcinoma HT-1376 cell lines were cultured in Eagle's Minimum Essential Medium (ATCC, Manassas Va.) supplemented with 10% Fetal Bovine Serum (Sigma Aldrich), 1% Penicillin/Streptomycin (Sigma Aldrich) and exposed to 5% CO₂ hepa-filtered air at 37 degrees Celsius. Cells were cultured to 80% confluence and collected using 0.25% trypsin, 0.02% EDTA solution at room temperature. Cell pellets were isolated and re-suspended in 100 μL D-PBS (Mg—, Ca—) per 1.0×10̂6 cells in preparation for inducing tumors in mice.

### Animals

Animal research was conducted in accordance with the guidelines by the Canadian Council on Animal Care. CB-17 white-haired Severe Combined Immuno-Deficient (SCID) male mice were obtained from Charles River Inc. (Wilmington, Mass., USA). A total cell volume of 1.0×10̂6 cells suspended in 100 μL of D-PBS (Mg—, Ca—) was injected subcutaneously to the lower right hind leg of the mouse and tumors were allowed to develop over a period of 2-3 weeks in order to reach a diameter of 5-7 mm for experiments.

### In Vivo Image Acquisition and Analysis

Blood flow was detected using a VEV0770 ultrasound unit (VisualSonics, Toronto, Ontario, Canada) in power (amplitude) Doppler mode with a 25 MHz transducer (Visualsonics RMV-710B, centre frequency=20 MHz, lateral resolution=149 μm, axial resolution=54 μm, (manufacturer specifications)). The real-time microvisualization transducer was employed to acquire 3D volumetric scans (scan speed 2.0 mm/s, wall filter 2.5 mm/s and step size of 0.2 mm, 90% bandwidth, 12.8 mm focal depth) where the center of the tumor was placed at the acoustic focus. Mice were anaesthetized using a combination of 2% oxygen ventilated isoflurane for induction and then with intraperitoneal injection of ketamine 100 mg/kg, xylazine 5 mg/kg, and acepromazine 1 mg/kg in 0.9% sodium chloride saline titrated at 0.02 mL intervals to a maximum dose of 0.1 mL. All mice were imaged before treatment administration in order to assess baseline microvessel blood flow for both acute and longitudinal studies. For “acute” studies, the mice were scanned 24 hours after treatment. For “longitudinal” studies, the mice were scanned at 24 hours, 7 days, 14 days, 21 days, and 28 days as established by animal health endpoints criteria. The number of images acquired per scan ranged from 60-100 frames depending on tumor size. Power Doppler images were converted into device-independent binary data for quantitative analysis using custom designed software with MATLAB (Mathworks Inc. Natick, Mass., USA). Microvessel flow signals were determined by selecting a region-of-interest (“ROI”) encompassing the whole tumor in each frame. Each frame was constructed by quantized color and non-color voxels. The vascular index (“VI”) was calculated by dividing the total number of color voxels (representing the power Doppler signal from red-blood cell backscatters) by the non-color voxels (representing non-vascular regions). The normalized vascular index (“NVI”) was calculated by comparing the vascular index at each time point against the baseline vascular index.

In order to minimize experimental variability, each experimental group of animals (treatment type) had imaging carried out within the same session to reduce variability. To minimize variability during acquisition settings, the tumor-bearing hind leg was immersed in a constant temperature water bath to couple the transducer to the tumor. The water used was degassed in advance through vacuum pressure prior to using for experiments to minimize air bubbles which interfere with imaging. Titrated anesthetic was administered in order to ensure that the animal's blood pressure was maintained and to also reduce any respiratory and cardiac motion artifacts on the Doppler signal. The mouse's body temperature was maintained to reduce vasoconstriction by keeping it on a heating pad with its core temperature monitored rectally. Animals used for experiments were similar in age, size and weight. Any data frames which had motion artifacts were removed from analysis.

### In Vivo Studies

Microbubbles and Ultrasound Activation: DEFINITY® Perflutren lipid microspheres (Lantheus Medical Imaging, N. Billerica Mass., USA) were shaken using a Lantheus device for 45 seconds at 3000 rpm. Low (1% v/v) and high (3% v/v) microbubble concentrations were calculated according to total mouse blood volume estimated by animal weight. The microbubbles were diluted in sterile normal saline and injected via the tail vein. A secondary injection (0.1 cc) of normal saline was used to flush the tail vein prior to treatment. Mice were mounted onto a custom stage and partially immersed into a 37 degree Celsius water bath for ultrasound exposures. The ultrasound therapy system involved a micro-positioning system, waveform generator (AWG520, Tektronix), power amplifier with pulser/receiver (RPR4000, Ritec), and a digital acquisition system (Acquiris CC103). Animals were exposed within the half maximum peak of the acoustic signal (−6 dB beam width of 31 mm and depth of field greater than 2 cm) 16-cycles tone burst at 500 kHz center frequency using a 2.85 cm unfocused planar ultrasound transducer (ValPey Fisher Inc, Hopkinton, Mass.) and at 3 kHz pulse repetition frequency for 50 ms at a time with a peak negative pressure set to 570 kPa, corresponding to a mechanical index (“MI”) of 0.8. An intermittent 1950 ms period between sonification was employed to minimize biological heating in the tissue during ultrasound exposures. The total insonification time was 750 ms over 5 minutes.

Irradiation: The tumors were X-irradiated 5 minutes after ultrasound treatment using an irradiation cabinet device (Faxitron, Wheeling Ill., USA). Doses of 0, 2, and 8 Gy were administered at a dose rate of 200 cGy/minute, 160 kVp energy and a source-skin distance (“SSD”) of 30 cm as per the specifications of the device. Corporal lead sheet shielding was used with a circular aperture to expose only the tumor.

### Histopatholocty

Tumors were fixed in 10% acetate buffered formalin (Fisher Scientific Canada, Ottawa Ontario, Canada) following excision at 24 hours (for acute studies) and 21-28 days (for long term studies) after treatment. Tumors were fixed at room temperature for 4 hours and then transferred to 4 degrees Celsius for 24 hours before processing using a Leica ASP300 smart tissue processor (Leica Microsystems, Richmond Hill, Ontario, Canada). Tissues were embedded in paraffin (Leica EG 1160, Leica Microsystems, Richmond Hill, Ontario, Canada) and prepared as 5 μm sections onto slides with standard hematoxylin and eosin (H&E) staining techniques used for both acute and longitudinal studies. Staining using TdT-mediated dUTP-biotin nick end-labeling (TUNEL) was used in acute studies to visualize apoptotic regions. Cluster of Differentiation-31 (CD31) staining was implemented to count and quantify endothelial cells within the tumor relative to the presence of intact and disrupted vessels.

Specifically, for microscopy of specimens on slides, a Leica DC100 microscope was used with a 20× objective coupled to a 1 MPixel Leica DC100 video camera wired to a 2 GHz PC running Leica IM1000 software (Leica GmbH, Germany). A normalized CD31 vascular index was calculated using 10 randomly selected regions of interest per tumor slice from 5 slices per animal tumor. The vascular index was calculated as the ratio of the summer intact luminal vessel number/area measured to the total vessel number/area measured (including intact luminal vessels and vessels which had been ruptured or collapsed by microbubble exposure). Vessels stained with CD31, in addition to areas of cell death, and all other microscopy measures were quantified in histology and immunohistochemistry tumor sections assisted by the use of Image-J (NIH, USA).

### Statistical Analysis

A detailed analysis was conducted using GRAPHPAD INSTAT (GraphPad Software Inc., La Jolla, USA) and a statistician was consulted to review the most appropriate statistical method. One-way ANOVA statistical analysis with Dunnett's Test or Tukey's multiple comparison's test was performed.

### Results

### Microbubbles alone effectively target vasculature in HT-1376 Bladder Cancer Xenografts In Vivo

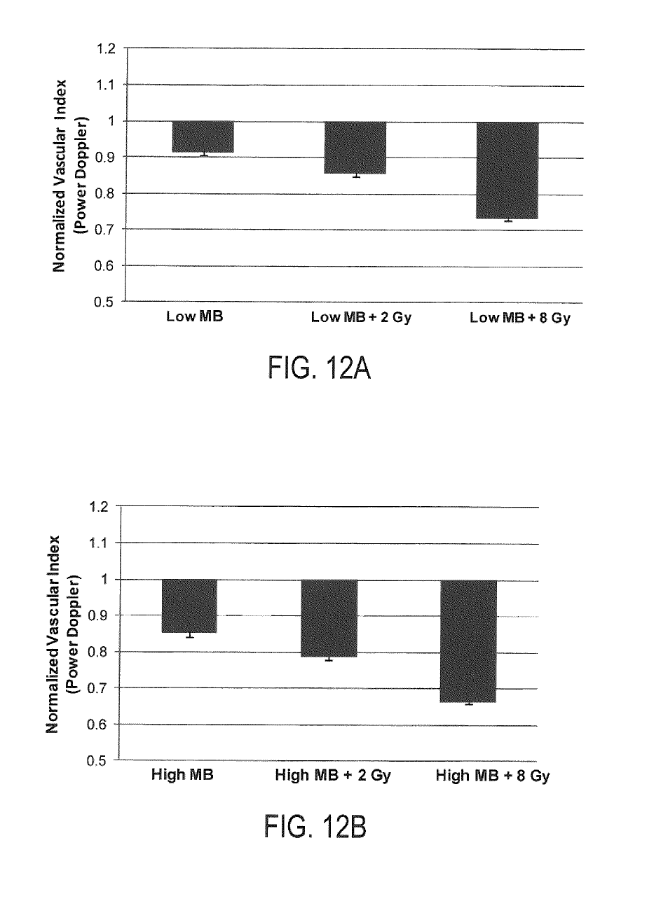

In order to quantify vasculature damage as a result of ultrasound-microbubble treatments alone, HT-1376 bladder cancer xenografts were treated with low and high concentrations of microbubbles and exposed to ultrasound positioned over the tumor. The acute studies revealed that HT-1376 bladder carcinoma microvessels were responsive to treatments and demonstrated a reduction in the mean power Doppler index from the initial measured baseline. Low concentration microbubble-ultrasound treatment after 24 hours revealed a normalized vascular index was 0.91±0.01 (SEM), representing a reduction in vasculature by approximately 8.6% (FIG. 12A). The higher microbubble-ultrasound treatment resulted in a normalized vascular index of 0.85±0.01 or a reduction in vasculature by 15% (FIG. 12B). The long term studies revealed similar results. Tumors that were treated with 1% and 3% microbubble concentrations when driven by ultrasound resulted in a mean power Doppler measurement of 0.71±0.01, and 0.70±0.01 respectively after 21 days.

### The Combination of Ultrasound-Driven Microbubbles and Radiation Synergistically Decreases Vasculature in HT-1376 Bladder Cancer tumors In Vivo

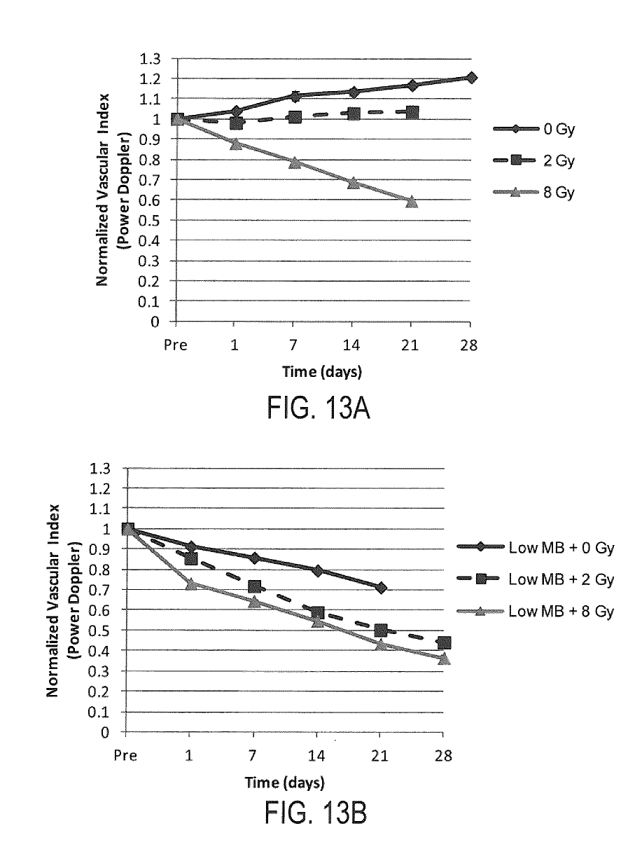

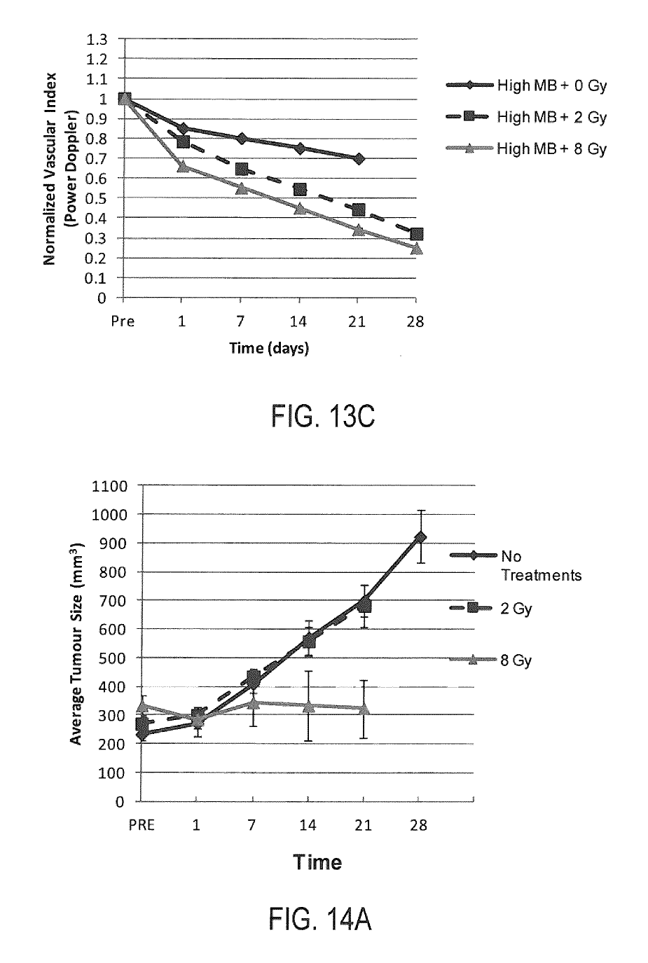

In order to explore how radiation alone affected the vasculature, HT-1376 bladder cancer tumors were exposed to 2 and 8 Gy of ionizing radiation as experimental controls. The findings revealed that the tumor vasculature was diminished in flow within the first 24 hours of treatment, primarily at the 8 Gy dose (NVI=0.88±0.00) and negligibly at the 2 Gy dose (NVI=0.98±0.00) (FIG. 13A). In our long term studies, a negligible detected increase in flow at 2 Gy was observed (NVI of 1.04±0.00) and the 8 Gy dose reached a normalized vascular index of 0.60±0.01 (FIG. 13A).

Results indicated that combination treatments with microbubbles and radiation lead to synergistic anti-vascular effects as observed by power Doppler and immunohistochemistry. Short term studies (24 h) demonstrated that at low microbubble concentration, there was a decrease in detectable flow with 2 Gy administration (NVI=0.86,±0.00) and with 8 Gy (NVI=0.73±0.00) compared to the baseline (FIG. 12B). The higher microbubble concentration revealed a further decrease in detectable blood flow when combined with 2 Gy (NVI=0.79±0.01) and 8 Gy (NVI=0.66±0.00) which was greater than with microbubbles alone (FIG. 12C).

Long term studies showed similar reduction in vascularity when measured at 21 days. When 2 Gy was combined with the low microbubble concentration treatment, an NVI of 0.50±0.01 was observed and at 8 Gy with low concentration microbubbles, an NVI of 0.43±0.00 was observed (FIG. 13B). When 2 Gy radiation was combined with high concentration microbubbles a NVI of 0.44±0.01 was observed. When 8 Gy radiation was administered with the high concentration of microbubbles in the presence of ultrasound a NVI of 0.34±0.01 was observed (FIG. 13C). These changes show significant synergistic treatment effects when compared to treatment of ultrasound-driven microbubbles alone.

### The Combination of Ultrasound Driven Microbubbles and Radiation Significantly Enhances Tumor Cell Death in the Tumor's Centre Compartment Caused by Endothelial Cell Disruption

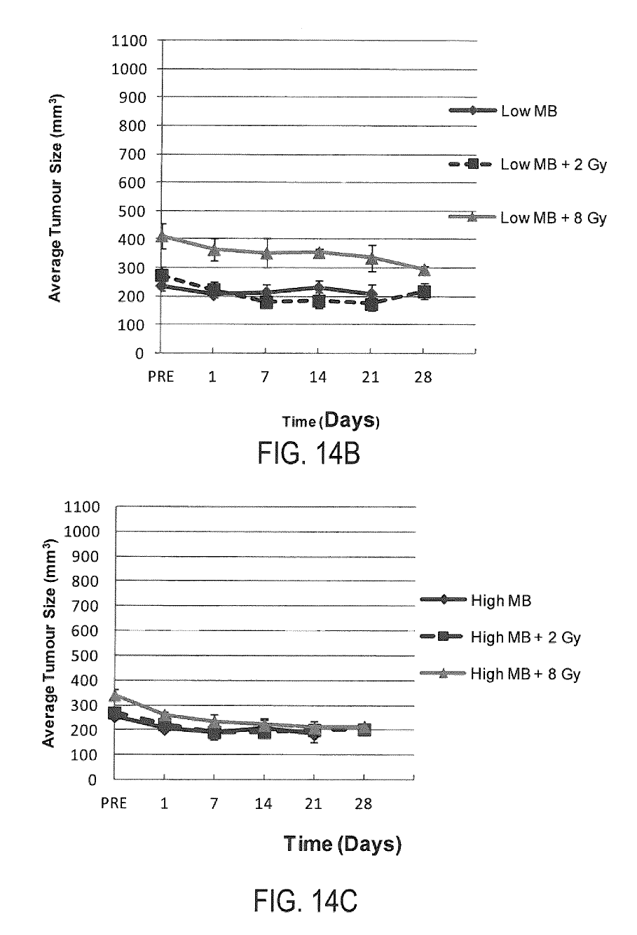

We tested to determine the efficacy of microbubbles in combination with radiation to induce vascular atrophy and to enhance tumor kill potential. We assessed tumor cell death using immunohistochemistry and measured changes in tumor size at each imaging time point. Treated tumors with combination therapy revealed tumor growth delay (FIGS. 14A-14C). Treated tumors demonstrated significant response to treatment within the tumor's center region. Circumferential tumor regions showed less significant signs of cell death according to the immunohistochemistry. Hematoxylin and eosin staining was performed on long term studies and demonstrated an increase in cell death as higher concentrations of ultrasound-driven microbubbles and radiation were combined together. Short term treated and untreated tumors revealed insignificant changes in tumor size; however, the effects of treatment were more obvious in the long term results. Short term and long term tumors showed elevated regions of cell death according to immunohistochemical (TUNEL) staining.

In order to validate treatment effects and confirm power Doppler effects, CD31 immunohistochemical analysis was performed. Power Doppler quantification and immunohistochemistry (CD31) results both demonstrated a decrease in measured vascularity. CD31 stains were used to detect presence of endothelial cells in all treated and non-treated groups. Vascular structures were identified and analyzed for the presence of viable endothelial cells. Analysis revealed decreased CD31 counts in treated tumors, corresponding with a reduction in the number of viable blood vessels found within the tumor.

This example demonstrates that ultrasound-mediated microbubble treatment of tumors can be an effective vascular targeting agent that can also potentiate the effects of radiation therapy in bladder cancer xenografts in vivo. Ultrasound-stimulated microbubbles in combination with radiation can induce rapid hematopoietic disruption, which presents as tumor cell death within the tumor center in both short and long-term studies.

Vascular targeting agents can be an effective partnering modality in eradicating the tumor stroma. The current study suggests that ultrasound-mediated microbubbles can be successfully used as a vascular targeting agent and can enhance radiosensitivity in bladder cancer carcinoma.

### EXAMPLE 4

### Cellular Characterization of Ultrasound-Stimulated Microbubble Radiation Enhancement

In this example, the inventors demonstrate that microbubble-stimulated radiation affect tumor vascularization and Ki-67 activity greater than radiation alone or ultrasound-stimulated microbubble treatment alone. The combined therapy resulted in the greatest destruction of tumor vasculature concomitant with the greatest detected extent of tumor cell death. The resultant tumor core exhibited hypoxia, but, paradoxically, with an enhancement of radiation induced cell death as assessed by immunohistochemistry and clonogenic cell survival assays.

### Materials and Methods

### Cell Culture

Prostate cancer cells (PC3, American Type Culture Collection, Manassas, Va., USA) were cultured in RPMI-1640 (Wisent Inc., St. Bruno, Canada) culture media which included 10% Fetal Bovine Serum (FBS) (Thermo Fisher Scientific, Waltham, USA) and 100 U/mL of Penicillin/Streptomycin (Invitrogen, Carlsbad, USA). Cells were grown and maintained under humidity at 37 degrees Celsius, 5% CO₂. Confluent cells were harvested using 0.05% Trypsin-EDTA (Invitrogen, Carlsbad, USA). Cells were collected by centrifugation at 4° C. for 10 min (200 g) and were re-suspended in phosphate buffer saline (PBS) in preparation for animal injection.

### Treatments

Five- to six-week old CB-17 Severe Combined Immuno-Deficiency (SCID) male mice (Charles River Laboratories International, Wilmington, Mass., USA) had xenograft tumors induced by injecting 1×10̂6 PC3 cells suspended in 50 μL of PBS subcutaneously in the upper hind legs of the animals. Tumors were allowed to develop to a diameter of 7-10 mm within approximately one month from the initial time of induction.

Animals were anaesthetized prior to treatment by an intraperitoneal injection of a mixture of Ketamine (100 mg/kg), Xylazine (5 mg/kg) and Acepromazine (1 mg/kg) (Sigma, Burlington, ON, Canada). The treatments included: radiation alone (0 Gy, 2 Gy, 8 Gy), ultrasound-stimulated microbubbles (0.3% v/v) alone and a combination of the ultrasound-microbubble treatments followed immediately by radiation. Eight animals were used per condition. DEFINITY® microbubbles (Lantheus Medical Imaging, N. Billerica Mass., USA) were activated by shaking for 45 seconds at 3000 rpm using a Lantheus Vial-shaker device.

The therapy set up included a wave-form generator (AWG520, Tektronix), an amplifier (RPR4000, Ritec), an acquisition system (Acquiris CC103) and an ultrasound transducer (central frequency of 500 kHz, ValpeyFisher Inc). The tumor on the hind leg was immersed into a 37 degree Celsius water bath and was positioned within the half maximum peak of the acoustic signal from the transducer. Tumors were exposed to 16 cycles tone burst of 500 kHz frequency with 3 kHz pulse repetition frequency for 5 minutes resulting in 750 milliseconds of exposure for an overall duty cycle of 0.25%. The peak negative acoustic pressure was 570 kPa (mechanical index of 0.8).

For radiation treatments, mice were shielded with a lead sheet, except for the tumor region, which was exposed to radiation through a confined circular aperture. A CP-160 cabinet X-radiator system (Faxitron X-ray Corporation, IL, USA) was used to deliver 0, 2, or 8 Gy at a rate of 200 cGy/minute. Animals were sacrificed 24 hours after treatment and tumors were harvested and fixed in 1% paraformaldehyde or embedded in optimal cutting temperature (OCT) media, then flash frozen in liquid nitrogen and stored at −80 degrees Celsius for future analyses.

### Clonogenic Assays

Excised tumor portions were mechanically and chemically dissociated as previously described elsewhere (Dow et al., Cancer Res. 42, 5262-5264 (1982)). Tumor cells were passed through a series of needles (18-, 20-, 22-gauge) and were then trypsinized with 0.25% trypsin at 37 degrees Celsius for 10-15 minutes. Media (RPMI-1640) supplemented with 10% FBS was then added and cells were washed twice by resuspension and centrifugation at 450 g. Cells were counted using a hemacytometer and 105 cells were plated in triplicate and incubated at 37 degrees Celsius and 5% CO₂ for 7 days to develop colonies. Colonies were then fixed and stained with 0.3% methylene blue/methanol for 20 minutes. The numbers of the counted colonies were compared and analysis by the Mann-Whitney test was used to determine the statistical significance.

### Histology and Immunhistochemistry

Specimens were fixed in freshly prepared 1% paraformaldehyde for up to 2 hours at room temperature then incubated at 4 degrees Celsius for 48 hours, after which fixative was replaced by 70% ethanol. Samples were then embedded in paraffin and 5 μm sections were placed on glass slides in preparation for staining. Histopathology was evaluated using H&E staining as well as ISEL and terminal dUTP nick end labeling (TUNEL) staining using the In Situ Apoptosis Detection Kit (R&D Systems; Minneapolis, USA), and was carried out according to manufacturer's instructions. Fragmented DNA was detected by labeled nucleotides (BrdU-labeled dNTPs) which were added to the 3′OH end by a terminal DNA transferase. Labeled tissue was incubated with BrdU antibody, then with Streptavidin-HRP, and the formed complex then labeled by TACS blue. The resulting dark blue nuclear staining was used to detect apoptotic nuclear chromatin. Nuclear Fast Red was used as a counter stain to stain all cells with normal cells stained pale pink, while apoptotic condensed cells stained red or purple. Areas of cell death within the sections of the tumors were measured using Image J (National Institutes of Health, Bethesda, Md., USA).

Immunolabeling was performed using a Histostain-plus kit (Invitrogen, Carlsbad, Calif.), following manufacturer's instructions. Tissue sections were deparaffinized in xylene and dehydrated in a graded ethanol series, and washed in PBS. In order to un-mask the antigenic sites, tissues were treated with 10 mM sodium citrate buffer and incubated at 95-100 degrees Celsius for 20-40 minutes. Tissues were left to cool at room temperature for 20 minutes. Slides were then washed with PBS and the endogenous peroxidase was quenched by 3% hydrogen peroxide in methanol. In order to block the non-specific background, 10% non-immune serum (goat) was used, which was followed by the incubation of the sections with the primary antibody for 1 hour at room temperature. A biotinylated secondary antibody was used, followed by incubation with horseradish peroxidase conjugated to streptavidin. This formed complex was then labeled by AEC creating an intense red color. Hematoxylin was used as a counterstain. All stained tissues were imaged using LEICA DM LB light microscope and Leica IM1000 software.

Primary antibodies (Abcam, Cambridge, Mass., USA) used for the immunostaining included polyclonal antibody against CD31 (mouse) used at 0.2 mg/ml, polyclonal against VEGF (human) at 0.5 mg/ml, polyclonal antibody against PHD2/prolyl hydroxylase (human) at 1 mg/ml and polyclonal antibody against Ki67 (human) at 1 mg/ml. All antibodies were used at a 1:20 dilution except for Ki67, which was used at a 1:10 dilution. Factor VIII and Gamma H2AX immunolabels were done by the Biomarker Imaging Research Laboratory at Sunnybrook Health Sciences Centre (Toronto, Canada).

In order to assess proliferation, Ki67 immunolabeling of cells was performed, Ki67-positive cells were counted throughout each tumor section and the total area of each section was then calculated to find the number of proliferating cells/mm². A vascular index with CD31 staining was similarly determined. The results were then averaged and compared using a Mann-Whitney test or t-test to determine statistical significance.

For ceramide staining, tumor tissues embedded in OCT medium were snap frozen in liquid nitrogen then stored at −80 degrees Celsius. Frozen, 8 micron sections were then prepared and used for ceramide labeling after washing the sections with PBS at room temperature. Immunolabeling was then carried out. For the immunolabelling of PHD2, VEGF, factor VIII, CD31, ceramide and Gamma H2AX, the quantification of the staining was done using either Image J (National Institutes of Health, Bethesda, Md., USA) (Immuno-Ratio) or a tally counter.

### Results

### Cell Death, Survival and Proliferation

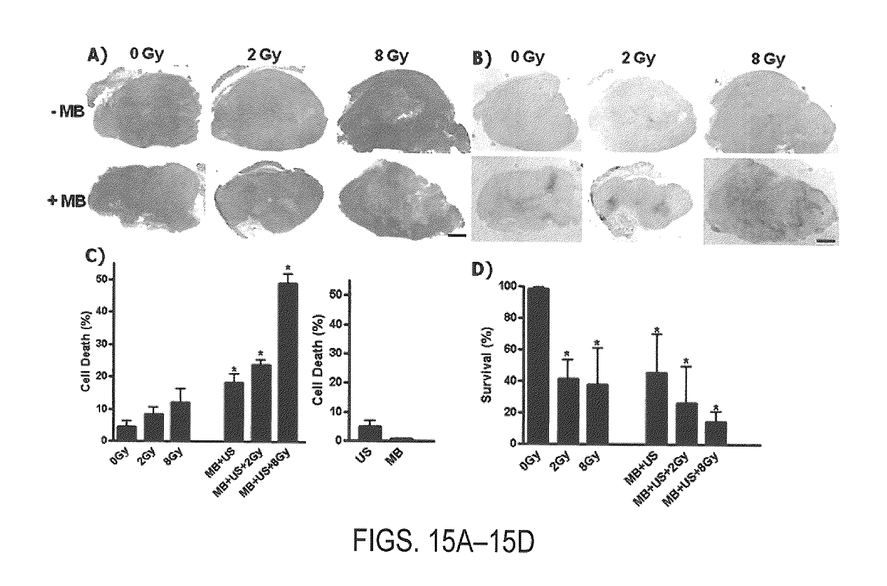

Results revealed increases in cell death with enhancement apparent when ultrasound-stimulated microbubble treatments (US+MB) were combined with either 2 Gy or 8 Gy radiation doses (US+MB+2 Gy, US+MB+8 Gy). Tumor disruption and cell death was apparent in hematoxylin and eosin (H&E) stained sections as a white blanched central area (FIG. 15A) with corresponding ISEL staining (FIG. 15B). Cell death approached 23.8±1.5% and 49.2±2.9% when ultrasound-stimulated microbubble treatment was combined with 2 Gy and 8 Gy radiation doses, respectively. On their own, radiation treatments caused minimal increases in cell death (FIG. 15C). Analyses indicated that treatment-induced cell death levels were significantly different when comparing the control with bubble-alone treatment (P<0.05) or when comparing the combined treatments with 2 Gy (P<0.029), or with 8 Gy (P<0.012) treatments alone. In contrast, single treatments of 2 Gy or 8 Gy did not reveal significant differences from the control. Control treatments with ultrasound in the absence of microbubbles, or microbubbles administered in the absence of ultrasound-stimulation caused no appreciable effect.

Clonogenic survival results for a single treatment (radiation alone, ultrasound-stimulated microbubbles alone, or the combination) are given in FIG. 15D. Results demonstrated that the combination of the ultrasound-stimulated microbubbles and radiation doses had less survival than either of the single modalities used for treatment alone. For the single treatments with 2 Gy, 8 Gy, or ultrasound-stimulated microbubbles, we observed cell survival ranging between 45.5±24.8% to 38.2±22.8%. Cell survival decreased with the combined treatments to 26.78±22.7% and 14.4±6.9% for the treatments with 2 Gy and 8 Gy, respectively. Data were significant when compared to the control (P<0.05). Statistical analyses using the Mann-Whitney test showed significant P values when compared to the control groups. A significant difference was found between the control and 2 Gy (P<0.008), 8 Gy (P<0.018), and US+MB (P<0.048) conditions. Differences were also present between the control and combined treatment of US+MB+2 Gy with P<0.018, or the treatment with US+MB+8 Gy with P<0.008. One-way ANOVA was also used demonstrating a significant change with P<0.0095 (FIG. 15D).

Higher magnification inspection of haematoxylin and eosin, and TUNEL stained tumor sections revealed that the combination of ultrasound-stimulated microbubble and radiation treatments induced cellular apoptosis. Prominent retraction artifact or areas of acellular destruction were evident with combination therapy exposure. Cellular damage was mostly confined to the center of the tumors or within defined tumor regions that seemed to be associated with vasculature. Histopathology indicated both mixed apoptotic and necrotic morphologies with cells exhibiting ruptured membranes (necrotic cells) as well as condensed and fragmented nuclear material (apoptotic cells). Results were generally consistent with colony assay data and indicated less cell survival with the combined treatments than with any of the single treatments.

In order to investigate the response of a number of essential biological processes that are necessary for the maintenance of tumor cells, factors such as cellular proliferation, vascular leakage, angiogenesis, hypoxia, and levels of DNA damage analysis were assessed using immunohistochemistry.

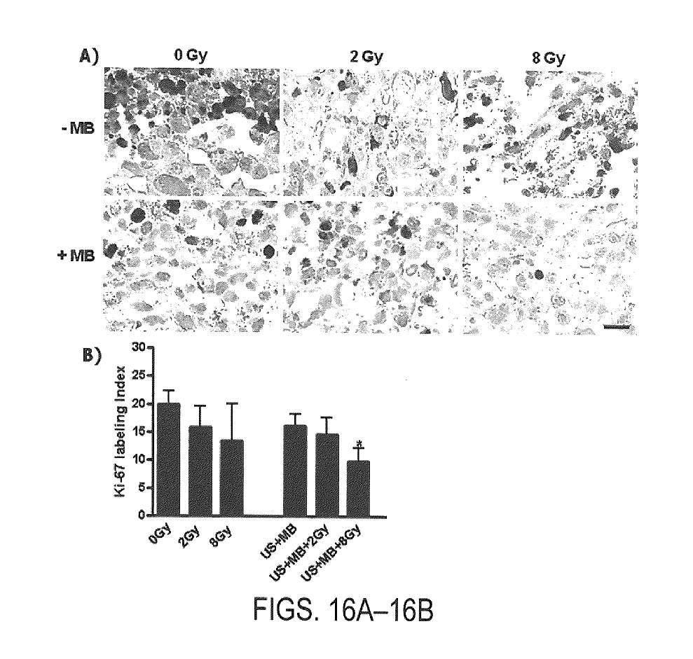

Investigation of tumor cell proliferation using anti-Ki67 antibodies, Ki67-a proliferation marker, assessed nuclear staining in tissues exposed to the different treatments. There was less nuclear staining for the combined treatments with either 2 Gy or 8 Gy, when compared to the control or to the other treatments (FIG. 16A). Statistical analysis using the Mann-Whitney test indicated that the combined treatments were significantly different from those of the controls. The 2 Gy treatment combined with ultrasound-stimulated microbubbles (US+MB+2 Gy) (15±3 Ki67+cells/mm²) compared to ultrasound-stimulated microbubble only (23±2 Ki67+cells/mm²) was statistically significantly different (P<0.033). Comparing the combined 8 Gy treatment (10±2 Ki67+cells/mm²) to the untreated control (20±2 Ki67+cells/mm²), resulted in a P<0.024, or comparison to the ultrasound-stimulated microbubble treatment (9US+MB+8 Gy) resulted in a P<0.004. The single treatments with radiation or ultrasound-stimulated microbubbles did not reveal significant differences from the controls (FIG. 16B).

### Treatment Effects on Vasculature, Oxygenation, Ceramide, and DNA Damage

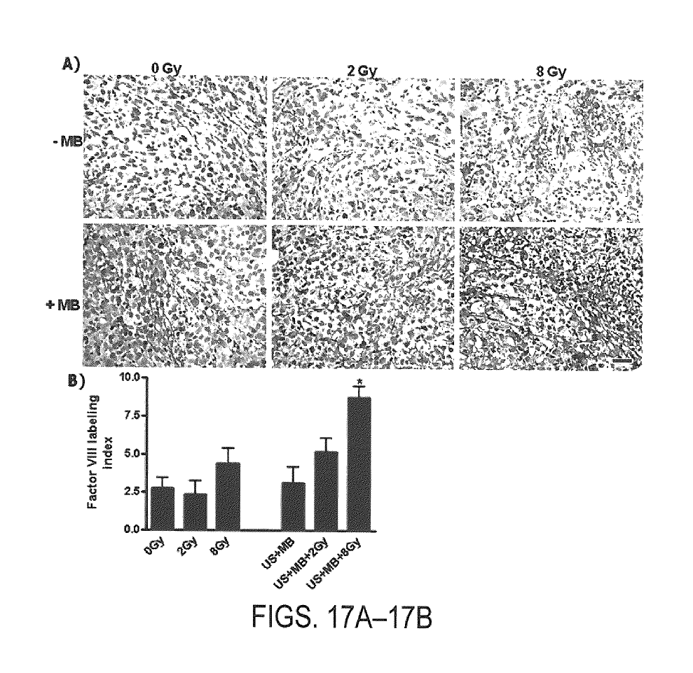

Tumor vascular damage associated with treatments was assessed by immunolabeling of clotting Factor VIII to evaluate the extent of disruption and the resulting blood leakage (FIGS. 17A and 17B). Increased vascular damage was observed which was associated with an increased vascular leakiness, predominantly associated with the combined treatment of ultrasound-stimulated microbubbles and 8 Gy (P<0.029).

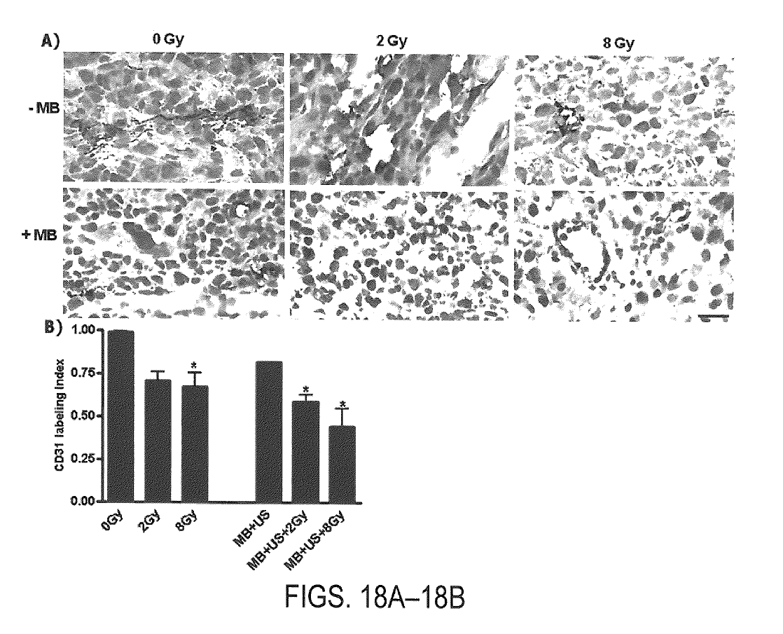

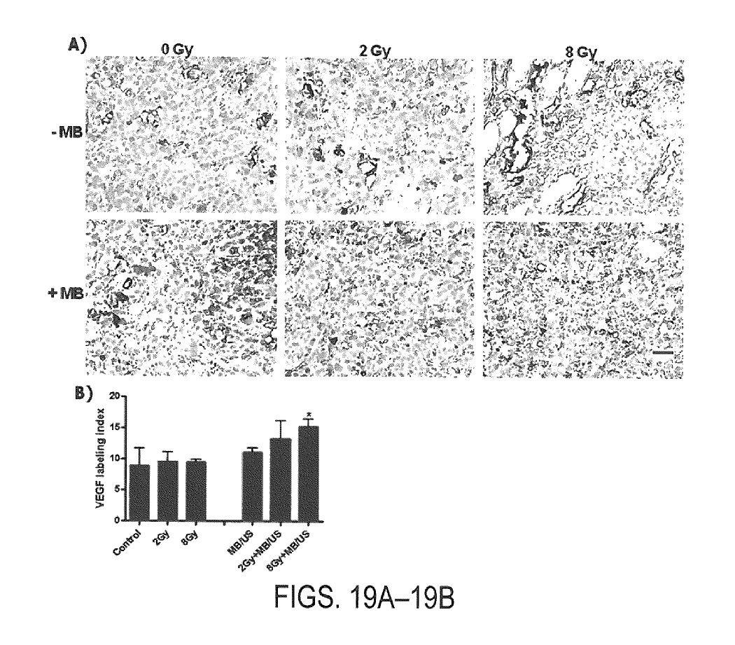

Changes in vascular index were investigated using CD31 immunohistochemistry, a cell surface receptor expressed on the membrane of endothelial cells considered as a marker to measure angiogenesis. Vascular labeling was significantly decreased when either 8 Gy (P<0.043), or ultrasound-stimulated microbubble and 2 Gy (P<0.032), or when the combined treatment with 8 Gy (P<0.01) were used (FIGS. 18A and 18B), as assessed using the t-test. In order to investigate treatment effects on angiogenesis signaling, VEGF was assessed using immunolabeling. A significant signaling increase was observed with the combined treatment with 8 Gy (P<0.032) (FIGS. 19A and 19B).

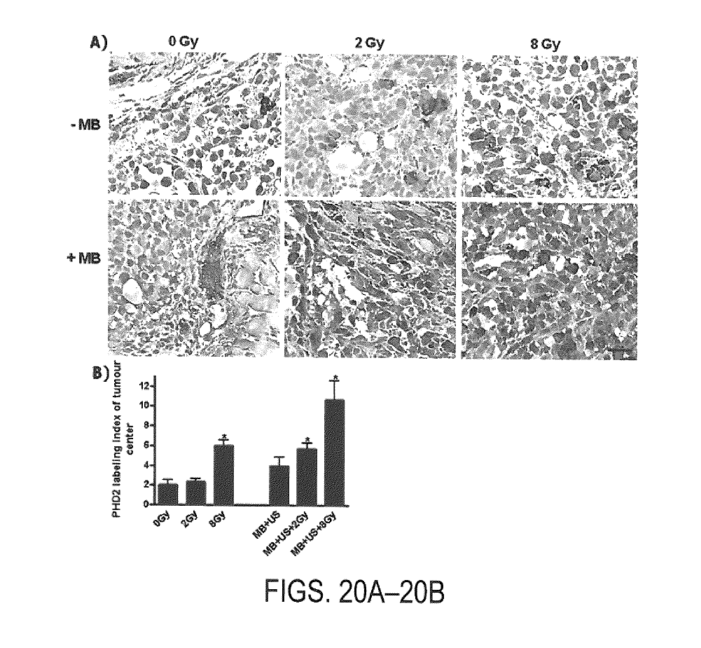

Since these vascular treatments may affect oxygenation of tissue, hypoxia was evaluated by staining for PHD2, an oxygen-sensing molecule that modulates hypoxia-inducible factor (HIF) response under low oxygen levels. Labeling of PHD2 was observed using immunohistochemistry in tumor cells and endothelial cells with exposure to different treatments (FIGS. 20A and 20B). An increase in the level of PHD2 in the center of the treated tumors was observed when treating with the higher radiation dose of 8 Gy (P<0.05), or with the ultrasound-stimulated treatments combined with radiation—MB+US+2 Gy (P<0.008), or MB+US+8 Gy (P<0.012).

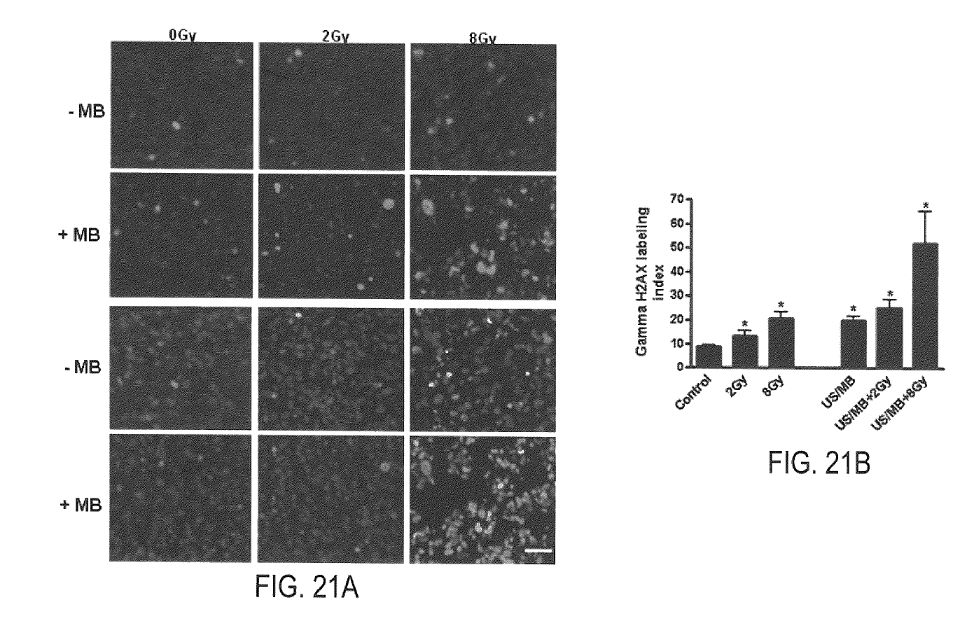

The effects of ionizing radiation were evaluated by staining with antibodies against Gamma H2AX (FIGS. 21A and 21B) which is a histone subtype associated with DNA damage. Immunolabeling of Gamma H2AX revealed significantly elevated levels of Gamma H2AX production under the different treatments (P<0.029, ultrasound-stimulated microbubbles alone and combined with 2 Gy, and P<0.014, for treatments with 2 Gy, 8 Gy and ultrasound-stimulated microbubbles and 8 Gy). A significant increase in Gamma H2AX was also observed when comparing single 2 Gy treatments to the combined treatment of ultrasound-stimulated microbubbles and 2 Gy (P<0.029) or when comparing 8 Gy to the combined therapy involving 8 Gy (P<0.014) with the combined treatments demonstrating more staining. This was further supported by one-way ANOVA testing with a P<0.002.

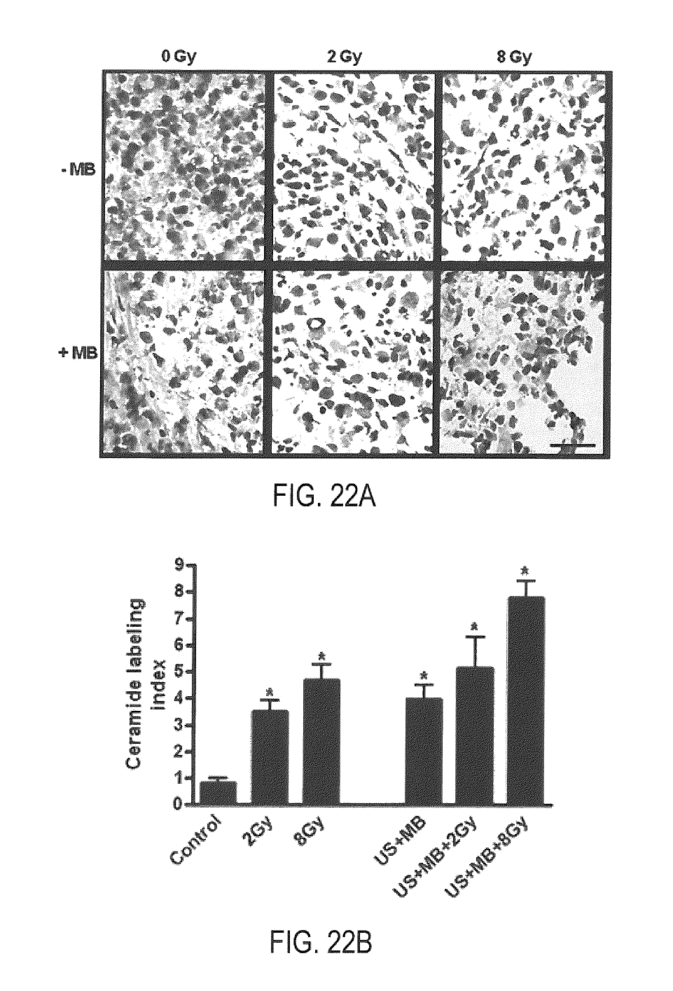

Findings (FIGS. 22A and 22B) indicated increases in ceramide with microbubble exposure and with radiation. Effects were greatest in the treatment with ultrasound-stimulated microbubbles and 8 Gy radiation exposure (P<0.05).

Results indicate microbubble-stimulated radiation affected tumor vascularization and Ki-67 activity greater than radiation alone or ultrasound-stimulated microbubble treatment alone. The combined therapy resulted in the greatest destruction of tumor vasculature concomitant with the greatest detected extent of tumor cell death. The resultant tumor core exhibited hypoxia but paradoxically, with an enhancement of radiation induced cell death as assessed by immunohistochemistry and clonogenic cell survival assays.

The combined treatments of ultrasound-activated microbubbles with radiation caused changes in tumor vasculature and the cellular microenvironment as demonstrated here through immunohistochemical staining analyses. Changes occurred in immunolabeling markers linked to cell death. Changes were also apparent in Ki67 linked to cellular proliferation. Vascular changes were also shown as detectable through CD31 labeling and Factor VIII staining. Changes were also apparent in Gamma H2AX in tumor cells showing an enhancement of effect induced by ultrasound-microbubble stimulation of endothelial cells leading to increased vascular destruction.

The present invention has been described in terms of one or more preferred embodiments, and it should be appreciated that many equivalents, alternatives, variations, and modifications, aside from those expressly stated, are possible and within the scope of the invention.

1. A method for controlling an ultrasound system to induce a therapeutic accumulation of ceramide in a target region of a subject, comprising:
directing the ultrasound system to expose a target region in the subject to which a microbubble agent has been provided to an effective exposure of ultrasound sufficient to induce a therapeutic accumulation of ceramide in the target region.

2. The method as recited in claim 1 in which the ultrasound system is directed to expose the target region to the ultrasound exposure only when a concentration of the microbubble agent in the target region is at a desired level that when exposed to the ultrasound exposure is sufficient to induce the therapeutic accumulation of ceramide in the target region.

3. The method as recited in claim 2 in which the concentration is in a range from about 1.8×10̂4 microbubbles per milliliter to about 5.4×10̂8 microbubbles per milliliter.

4. The method as recited in claim 2 in which the ultrasound exposure includes repeatedly exposing the target region to ultrasound for a duration of time followed by a time interval in which the target region is not exposed to ultrasound, the time interval being selected to allow the concentration of the microbubble agent to reach the desired level.

5. A method for inducing a therapeutic accumulation of ceramide in a target region of a subject, comprising:
administering an effective amount of a microbubble agent to a subject; and
directing an ultrasound system to expose a target region in the subject in which the microbubble agent is present to an effective exposure of ultrasound, whereby a therapeutic accumulation of ceramide in the target region is provided.

6. The method as recited in claim 5 further comprising exposing the target region to an effective dose of radiation sufficient to further increase ceramide accumulation in the cells in the target region.

7. The method as recited in claim 6 in which the dose of radiation is provided by at least one of an external beam source, a brachytherapy source, and a radioisotope source.

8. The method as recited in claim 6 in which the effective dose of radiation is provided at least one of contemporaneously with the ultrasound exposure and a selected time interval after the ultrasound exposure.

9. The method as recited in claim 8 in which the time interval is in a range from about five minutes to about twenty-four hours after the ultrasound exposure.

10. The method as recited in claim 9 in which the time interval is in a range from about three hours to about twelve hours after the ultrasound exposure.

11. The method as recited in claim 5 further comprising intravenously administering the microbubble agent to the patient such that the microbubble agent is provided to the target region.

12. The method as recited in claim 5 in which the administered microbubble agent is composed of microbubbles.

13. The method as recited in claim 5 in which the administered microbubble agent is composed of an exogenous material that produces microbubbles when exposed to ultrasound.

14. The method as recited in claim 13 in which the exogenous material includes at least one of perfluorocarbon liquid droplets and nanoemulsions.

15. The method as recited in claim 5 in which the microbubble agent is a targeted microbubble agent.

16. The method as recited in claim 15 in which the targeted microbubble agent is a vascular endothelial growth factor receptor-targeted microbubble agent.

17. A method for treatment of cancer in a subject, comprising:
administering an effective amount of a microbubble agent to the subject; and
providing an effective exposure of ultrasound to the subject sufficient to interact with the microbubble agent and cause vascular disruption in endothelial cells associated with a cancerous tumor, whereby a treatment of cancer in the subject is provided.

18. The method as recited in claim 17 further comprising providing an effective dose of an energy source to the cancerous tumor sufficient to increase the vascular disruption in the endothelial cells associated with the cancerous tumor.

19. The method as recited in claim 18 in which the energy source includes at least one of a radiation source, a thermal energy source, and an electromagnetic energy source.

20. The method as recited in claim 19 in which the radiation source is at least one of an external beam source, a brachytherapy source, and a radioisotope source.

21. The method as recited in claim 19 in which the thermal energy source is a cryoablation source.

22. The method as recited in claim 19 in which the electromagnetic energy source is at least one of a radio frequency ablation source, a microwave ablation source, and a laser ablation source.

23. An intravenously administrable composition for increasing ceramide in a population of cells associated with a tumor, comprising a microbubble agent having a concentration in the range of about 1.8×10̂4 microbubbles per milliliter to about 5.4×10̂8 microbubbles per milliliter, which when exposed to an effective exposure of ultrasound induces a therapeutic accumulation of ceramide in a subject.

24. The intravenously administrable composition as recited in claim 23 in which the microbubble agent comprises gas-filled microbubbles encapsulated in a shell composed of at least one of lipid and protein.

25. The intravenously administrable composition as recited in claim 24 in which the gas-filled microbubbles are composed of a fluorocarbon gas.

26. An ultrasound system for generating an anti-angiogenic or anti-tumor bioeffect in a population of cells located within a target region, comprising:
an ultrasound transducer configured to deliver ultrasound waves to a target region;
a control system in communication with the ultrasound transducer and configured to:
set an effective ultrasound exposure sufficient to induce disruption of an effective amount of a microbubble agent such that the disruption of the microbubble agent is capable of generating at least one of an anti-angiogenic and an anti-tumor bioeffect in cells; and
direct the ultrasound transducer to produce ultrasound waves at the set effective ultrasound exposure such that the ultrasound waves are delivered to the target region.
